(19) United States
(12) Patent Application Publication
(10) Pub. No.: US 2003/0224025 A1
(43) Pub. Date: Dec. 4, 2003

(54) Skin cosmetic formulations

(76) Inventors: Michael Gotsche, Mannheim (GB); Claudia Wood, Weinheim (DE); Reinhold Dieing, Schifferstadt (DE); Axel Jentzsch, Mannheim (DE)

Correspondence Address: Keil & Weinkauf, 1101 Connecticut Avenue N W, Washington, DC, 20036, US

(21) Appl. No.: 10/362,059

(22) PCT Filed: Aug. 16, 2001
(86) PCT No.: PCT/EP01/09438

(30) Foreign Application Priority Data
Aug. 22, 2000 (DE) 100412203

(51) Int. Cl. A61K 6/00

(52) U.S. Cl. 424/401

(57) ABSTRACT

The use of polymers obtainable by free-radical polymerization of

a) at least one vinyl ester of C₁-C₂₄-carboxylic acids in the presence of

b) polyether-containing compounds having a molecular weight of at least 300, and

c) optionally one or more further copolymerizable monomers in skin cosmetic formulations and in decorative cosmetics.

The present invention relates to aqueous or aqueous/alcoholic skin cosmetic formulations comprising, as film formers, polymers prepared by polymerization of vinyl esters and optionally further free-radical copolymerizable monomers in the presence of a polyether-containing compound.

Graft polymers of polyvinyl alcohol on polyalkylene glycols are already known.

DE 1 077 430 describes a process for the preparation of graft polymers of vinyl esters on polyalkylene glycols.

DE 1 094 457 and DE 1 081 229 describe processes for the preparation of graft polymers of polyvinyl alcohol on polyalkylene glycols by hydrolysis of the vinyl esters and their use as protective colloids, water-soluble packaging films, as sizes and finishes for textiles and in cosmetics.

DE 199 07587.5 describes the use of graft polymers of vinyl esters on polyethers for hair cosmetics.

A number of requirements are placed on skin cosmetic formulations:

they should be waterproof or water-resistant,

they should be transferable onto textiles and clothing to as low an extent as possible,

they should distribute easily on the regions of the body treated therewith (skin, eyelashes, lips, eyebrows, eyelids),

they should have a moisture-binding action,

they should convey a pleasant feel on the skin,

they should not have stickiness following application.

It is an object of the present invention to provide skin cosmetic formulations containing film-forming polymers which satisfy at least one of the above requirements in a particularly good manner and thus enable improved skin care.

We have found that this object is achieved according to the invention by using polymers obtainable by free-radical polymerization of

a) at least one vinyl ester in the presence of

b) polyether-containing compounds and optionally at least one further copolymerizable monomer c) in skin cosmetic formulations.

In the preparation of the polymers used according to the invention, it is possible for grafting onto the polyether-containing compounds (b) to result during the polymerization, which may lead to the advantageous properties of the polymers. However, mechanisms other than grafting are also conceivable.

Depending on the degree of grafting, the polymers used according to the invention are understood as meaning pure graft polymers and also mixtures of the abovementioned graft polymers with nongrafted polyether-containing compounds and homo- or copolymers of the monomers a) and c).

Polyether-containing compounds (b) which can be used are either polyalkylene oxides, based on ethylene oxide, propylene oxide, butylene oxide and other alkylene oxides, or polyglycerol. Depending on the type of monomer building blocks, the polymers contain the following structural units.

—(CH₂)₂—O—, —(CH₂)₃—O—, —(CH₂)₄—O—, —CH₂—CH(R⁶)—O—, —CH₂—CHOR⁷—CH₂—O—

where

R⁶ is C₁-C₂₄-alkyl;

R⁷ is hydrogen, C₁-C₂₄-alkyl, R⁶—C(═O)—, R⁶—NH—C(═O)—.

The structural units can either be homopolymers or random copolymers and block copolymers.

As polyethers (b), preference is given to using polymers of the formula I,

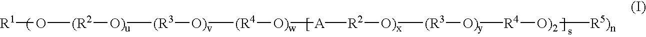

in which the variables independently of one another have the following meanings:

R¹ is hydrogen, C₁-C₂₄-alkyl, R⁶—C(═O)—, R⁶—NH—C(═O)—, polyalcohol radical;

R⁵ is hydrogen, C₁-C₂₄-alkyl, R⁶—C(═O)—, R⁶—NH—C(═O)—;

R² to R⁴ are

—(CH₂)₂—, —(CH₂)₃—, —(CH₂)₄—, —CH₂—CH(R⁶)—, —CH₂—CHOR⁷—CH₂—;

R⁶ is C₁-C₂₄-alkyl;

R⁷ is hydrogen, C₁-C₂₄-alkyl, R⁶—C(═O)—, R⁶—NH—C(═O)—;

A is —C(═O)—O, —C(═O)—B—C(═O)—O, —C(═O)—NH—B—NH—C(═O)—O;

B is —(CH₂)ₜ—, arylene, optionally substituted;

n is 1 to 1 000;

s is 0 to 1 000;

t is 1 to 12;

u is 1 to 5 000;

v is 0 to 5 000;

w is 0 to 5 000;

x is 0 to 5 000;

y is 0 to 5 000;

z is 0 to 5 000.

The terminal primary hydroxyl groups of the polyethers prepared on the basis of polyalkylene oxides, and the secondary OH-groups of polyglycerol can in this connection either be present freely in unprotected form, or be etherified with alcohols of chain length C₁-C₂₄ or esterified with carboxylic acids of chain length C₁-C₂₄, or reacted with isocyanates to give urethanes.

Alkyl radicals which may be mentioned for R¹ and R⁵ to R⁷ are branched or unbranched C₁-C₂₄-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethyl propyl, 1-ethyl-1-methylpropyl, 1-ethyl-2-methylpropyl, n-heptyl, 2-ethylhexyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Preferred representatives of the abovementioned alkyl radicals which may be mentioned are branched or unbranched C₁-C₁₂-, particularly preferably C₁-C₆-alkyl chains.

The molecular weight of the polyethers is at least 300 (according to number average), preferably in the range from 300 to 100 000, particularly preferably in the range from 500 to 50 000, very particularly preferably in the range from 800 to 40 000.

Homopolymers of ethylene oxide or copolymers with an ethylene oxide content of from 40 to 99% by weight are advantageously used. For the ethylene oxide polymers to be used in preference, the content of copolymerized ethylene oxide is thus from 40 to 100 mol %. Suitable comonomers for these copolymers are propylene oxide, butylene oxide and/or isobutylene oxide. Suitable examples are copolymers of ethylene oxide and propylene oxide, copolymers of ethylene oxide and butylene oxide, and copolymers of ethylene oxide, propylene oxide and at least one butylene oxide. The ethylene oxide content of the copolymers is preferably 40 to 99 mol %, the propylene oxide content is 1 to 60 mol % and the content of butylene oxide in the copolymers is 1 to 30 mol %. As well as straight-chain homo- or copolymers, it is also possible to use branched homo- or copolymers as polyether-containing compounds b).

Branched polymers can be prepared by, for example, adding ethylene oxide and optionally also propylene oxide and/or butylene oxides to polyalcohol radicals, e.g. to pentaerythritol, glycerol, or to sugar alcohols such as D-sorbitol and D-mannitol, but also to polysaccharides such as cellulose and starch. Within the polymer, the alkylene oxide units can be randomly distributed or be in the form of blocks.

It is, however, also possible to use polyesters of polyalkylene oxides and aliphatic or aromatic dicarboxylic acids, e.g. oxalic acid, succinic acid, adipic acid and terephthalic acid having molar masses of from 1 500 to 25 000, as described, for example, in EP-A-0 743 962, as polyether-containing compound. In addition, it is also possible to use polycarbonates by reaction of polyalkylene oxides with phosgene or carbonates such as, for example, diphenyl carbonate, and polyurethanes by reaction of polyalkylene oxides with aliphatic and aromatic diisocyanates.

Particularly preferred polyethers (b) are polymers of the formula I having an average molecular weight of from 300 to 100 000 (according to the number average), in which the variables independently of one another have the following meanings:

R¹ is hydrogen, C₁-C₁₂-alkyl, R⁶—C(═O)—, R⁶—NH—C(═O)—, polyalcohol radical;

R⁵ is hydrogen, C₁-C₁₂-alkyl, R⁶—C(═O)—, R⁶—NH—C(═O)—;

R² to R⁴ are

—(CH₂)₂—, —(CH₂)₃—, —(CH₂)₄—, —CH₂—CH(R⁶)—, —CH₂—CHOR⁷—CH₂—;

R⁶ is C₁-C₁₂-alkyl;

R⁷ is hydrogen, C₁-C₁₂-alkyl, R⁶—C(═O)—, R⁶—NH—C(═O)—;

n is 1 to 8;

s is 0;

u is 2 to 2 000;

v is 0 to 2 000;

w is 0 to 2 000.

Very particularly preferred polyethers b) are polymers of the formula I having an average molecular weight of from 500 to 50 000 (according to the number average), in which the variables independently of one another have the following meanings:

R¹ is hydrogen, C₁-C₆-alkyl, R⁶—C(═O)—, R⁶—NH—C(═O)—;

R⁵ is hydrogen, C₁-C₆-alkyl, R⁶—C(═O)—, R⁶—NH—C(═O)—;

R² to R⁴ are

—(CH₂)₂—, —(CH₂)₃—, —(CH₂)₄—, —CH₂—CH(R⁶)—, —CH₂—CHOR⁷—CH₂—;

R⁶ is C₁-C₆-alkyl;

R⁷ is hydrogen, C₁-C₆-alkyl, R⁶—C(═O)—, R⁶—NH—C(═O)—; n is 1;

s is 0;

u is 5 to 500;

v is 0 to 500;

w is 0 to 500.

However, the polyethers may also be silicone derivatives. Suitable silicone derivatives are the compounds known under the INCI name dimethicone copolyols or silicone surfactants, such as, for example, those available under the trade names Abil® (T. Goldschmidt), Alkasil® (Rhône-Poulenc), Silicone Polyol Copolymer® (Genesee), Belsil® (Wacker), Silwet® (Witco, Greenwich, Conn., USA) or Dow Corning (Dow Corning). These include compounds with the CAS numbers 64365-23-7; 68937-54-2; 68938-54-5; 68937-55-3.

Silicones are generally used in skin cosmetics to improve the feel of the skin. The use of polyether-containing silicone derivatives as polyether (b) in the polymers according to the invention can therefore additionally lead to an improvement in the feel of the skin.

Preferred representatives of such polyether-containing silicone derivatives are those which contain the following structural elements:

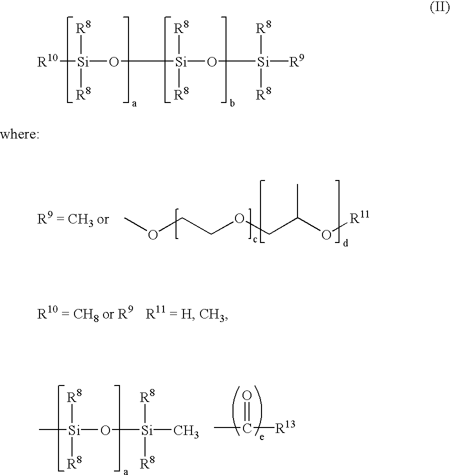

R¹³ is a C₁-C₄₀ organic radical which can contain amino, carboxylic acid or sulfonate groups, or for the case e=0, is also the anion of an inorganic acid,

and where the radicals R⁸ can be identical or different, and originate either from the group of aliphatic hydrocarbons having 1 to 20 carbon atoms, are cyclic aliphatic hydrocarbons having 3 to 20 carbon atoms, are of an aromatic nature or are identical to R¹², where:

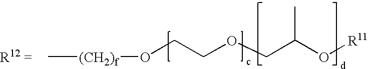

with the proviso that at least one of the radicals R⁸, R⁹ or R¹⁰ is a polyalkylene oxide-containing radical as defined above, and f is an integer from 1 to 6,

a and b are integers such that the molecular weight of the polysiloxane block is between 300 and 30 000,

c and d can be integers between 0 and 50, with the proviso that the sum c+d is greater than 0, and e is 0 or 1.

Preferred radicals R⁹ and R¹² are those in which the sum c+d is between 5 and 30.

The groups R⁸ are preferably chosen from the following group: methyl, ethyl, propyl, butyl, isobutyl, pentyl, isopentyl, hexyl, octyl, decyl, dodecyl and octadecyl, cycloaliphatic radicals, specifically cyclohexyl, aromatic groups, specifically phenyl or naphthyl, mixed aromatic-aliphatic radicals such as benzyl or phenylethyl and tolyl and xylyl and R¹².

Particularly suitable radicals R¹¹ are those in which in the case where R¹¹=—(CO)ₑ—R¹³, R¹³ is any desired alkyl, cycloalkyl or aryl radical which has between 1 and 40 carbon atoms and which can carry other ionogenic groups such as NH₂, COOH, SO₃H.

Preferred inorganic radicals R¹³ are, for the case e=0, phosphate and sulfate.

Particularly preferred polyether-containing silicone derivatives are those of the structure:

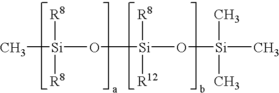

In addition, homo- and copolymers of polyalkylene oxide-containing ethylenically unsaturated monomers, such as, for example, polyalkylene oxide (meth)acrylates, polyalkylene oxide vinyl ethers, polyalkylene oxide (meth)acrylamides, polyalkylene oxide allylamides or polyalkylene oxide vinylamides can also be used as polyethers (b). It is of course also possible to use copolymers of such monomers with other ethylenically unsaturated monomers.

As polyether-containing compounds b), it is, however, also possible to use reaction products of polyethyleneimines with alkylene oxides. In this case, the alkylene oxides used are preferably ethylene oxide, propylene oxide, butylene oxide and mixtures thereof, particularly preferably ethylene oxide. Polyethyleneimines which can be used are polymers having number-average molecular weights of from 300 to 20 000, preferably from 500 to 10 000, very particularly preferably from 500 to 5 000. The weight ratio between used alkylene oxide and polyethyleneimine is in the range from 100:1 to 0.1:1, preferably in the range from 50:1 to 0.5:1, very particularly preferably in the range from 20:1 to 0.5:1.

For the polymerization in the presence of the polyethers b), the following free-radically polymerizable monomers may be mentioned as component a):

Vinyl esters of aliphatic, saturated or unsaturated C₁-C₂₄ carboxylic acids, such as, for example, formic acid, acetic acid, propionic acid, butyric acid, valeric acid, isovaleric acid, caproic acid, caprylic acid, capric acid, undecylenic acid, lauric acid, myristic acid, palmitic acid, palmitoleic acid, stearic acid, oleic acid, arachidic acid, behenic acid, lignoceric acid, cerotinic acid and melissic acid.

Preference is given to using vinyl esters of the abovementioned C₁-C₁₂ carboxylic acids, in particular of C₁-C₆ carboxylic acids. Vinyl acetate is very particularly preferred.

It is, of course, also possible to copolymerize mixtures of the respective monomers from group a).

The vinyl esters (a) can in addition also be used in admixture with one or more ethylenically unsaturated copolymerizable comonomers (c), where the content of these additional monomers should be limited to a maximum of 50% by weight. Preference is given to contents of from 0 to 20% by weight. The term ethylenically unsaturated means that the monomers have at least one free-radically polymerizable carbon-carbon double bond which can be mono-, di-, tri- or tetrasubstituted.

The preferred ethylenically unsaturated comonomers (c) additionally used can be described by the following formula:

X—C(O)CR¹⁵═CHR¹⁴

where

X is chosen from the group of radicals —OH, —OM, —OR¹⁶, NH₂, —NHR¹⁶, N(R¹⁶)₂;

M is a cation chosen from the group consisting of: Na⁺, K⁺, Mg⁺⁺, Ca⁺⁺, Zn⁺⁺, NH₄⁺, alkyl ammonium, dialkylammonium, trialkylammonium and tetraalkylammonium;

the radicals R¹⁶ can be identical or different and chosen from the group consisting of —H, C₁-C₄₀ linear or branched alkyl radicals, N,N-dimethylaminoethyl, 2-hydroxyethyl, 2-methoxyethyl, 2-ethoxyethyl, hydroxypropyl, methoxypropyl or ethoxypropyl.

R¹⁵ and R¹⁴ are independently of one another chosen from the group consisting of: —H, C₁-C₈ linear or branched alkyl chains, methoxy, ethoxy, 2-hydroxyethoxy, 2-methoxyethoxy and 2-ethoxyethyl.

Representative but nonlimiting examples of suitable monomers (c) are, for example, acrylic acid or methacrylic acid and salts, esters and amides thereof. The salts can be derived from any desired nontoxic metal, ammonium or substituted ammonium counterions.

The esters can be derived from C₁-C₄₀ linear, C₃-C₄₀ branched or C₃-C₄₀ carbocyclic alcohols, from polyfunctional alcohols having from 2 to about 8 hydroxyl groups, such as ethylene glycol, hexylene glycol, glycerol and 1,2,6-hexanetriol, from aminoalcohols or alcohol ethers such as methoxyethanol and ethoxyethanol, (alkyl)polyethylene glycols, (alkyl)polypropylene glycols or ethoxylated fatty alcohols, for example C₁₂-C₂₄-fatty alcohols reacted with 1 to 200 ethylene oxide units.

Also suitable are N,N-dialkylaminoalkyl acrylates and methacrylates and N-dialkylaminoalkylacryl- and -methacrylamides of the formula (III)

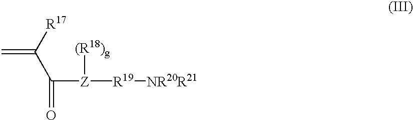

where

R¹⁷=H, alkyl having from 1 to 8 carbon atoms,

R¹⁸=H, methyl,

R¹⁹=alkylene having from 1 to 24 carbon atoms, optionally substituted by alkyl,

R²⁰, R²¹=C₁-C₄₀ alkyl radical,

Z=nitrogen when g=1, or oxygen when g=0.

The amides can be unsubstituted, N-alkyl- or N-alkylamino-monosubstituted or N,N-dialkyl-substituted or N,N-dialkylamino-disubstituted, where the alkyl or alkylamino groups are derived from C₁-C₄₀ linear, C₃-C₄₀ branched, or C₃-C₄₀ carbocyclic units. In addition, the alkylamino groups can be quaternized.

Preferred comonomers of the formula III are N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N-[3-(dimethylamino)propyl]methacrylamide and N-[3-(dimethylamino)propyl]acrylamide.

Comonomers (c) which can likewise be used are substituted acrylic acids and salts, esters and amides thereof, where the substituents on the carbon atoms are in the two or three position of the acrylic acid, and are independently of one another chosen from the group consisting of C₁-C₄-alkyl, —CN, COOH, particularly preferably methacrylic acid, ethacrylic acid and 3-cyanoacrylic acid. These salts, esters and amides of these substituted acrylic acids can be chosen as described above for the salts, esters and amides of acrylic acid.

Other suitable comonomers (c) are allyl esters of C₁-C₄₀ linear, C₃-C₄₀ branched or C₃-C₄₀ carbocyclic carboxylic acids, vinyl or allyl halides, preferably vinyl chloride and allyl chloride, vinyl ethers, preferably methyl, ethyl, butyl or dodecyl vinyl ether, vinylformamide, vinylmethylacetamide, vinylamine; vinyllactams, preferably vinylpyrrolidone and vinylcaprolactam, vinyl- or allyl-substituted heterocyclic compounds, preferably vinylpyridine, vinyloxazoline and allylpyridine.

Also suitable are N-vinylimidazoles of the formula IV, in which R²² to R²⁴ independently of one another are hydrogen, C₁-C₄-alkyl or phenyl:

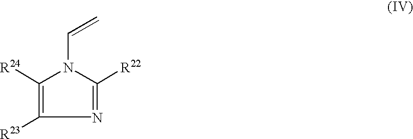

Other suitable comonomers (c) are diallylamines of the formula (V)

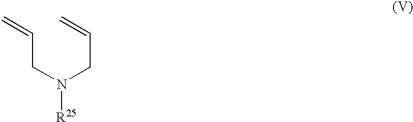

where R²⁵=C₁- to C₂₄-alkyl.

Other suitable comonomers (c) are vinylidene chloride; and hydrocarbons having at least one carbon-carbon double bond, preferably styrene, alpha-methylstyrene, tert-butylstyrene, butadiene, isoprene, cyclohexadiene, ethylene, propylene, 1-butene, 2-butene, isobutylene, vinyltoluene, and mixtures of these monomers.

Particularly suitable comonomers (c) are acrylic acid, methacrylic acid, ethyl acrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, n-butyl ethacrylate, isobutyl ethacrylate, t-butyl ethacrylate, 2-ethylhexyl ethacrylate, decyl ethacrylate, stearyl (meth)acrylate, 2,3-dihydroxypropyl acrylate, 2,3-dihydroxypropyl methacrylate, 2-hydroxyethyl acrylate, hydroxypropyl acrylates, 2-hydroxyethyl methacrylate, 2-hydroxyethyl ethacrylate, 2-methoxyethyl acrylate, 2-methoxyethyl methacrylate, 2-methoxyethyl ethacrylate, 2-ethoxyethyl methacrylate, 2-ethoxyethyl ethacrylate, hydroxypropyl methacrylates, glyceryl monoacrylate, glyceryl monomethacrylate, polyalkylene glycol (meth)acrylates, unsaturated sulfonic acids such as, for example, acrylamidopropanesulfonic acid;

acrylamide, methacrylamide, ethacrylamide, N-methylacrylamide, N,N-dimethylacrylamide, N-ethylacrylamide, N-isopropylacrylamide, N-butylacrylamide, N-t-butylacrylamide, N-octylacrylamide, N-t-octylacrylamide, N-octadecylacrylamide, N-phenylacrylamide, N-methylmethacrylamide, N-ethylmethacrylamide, N-dodecylmethacrylamide, 1-vinylimidazole, 1-vinyl-2-methylvinylimidazole, N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N,N-dimethylaminobutyl (meth)acrylate, N,N-diethylaminobutyl (meth)acrylate, N,N-dimethylaminohexyl (meth)acrylate, N,N-dimethylaminooctyl (meth)acrylate, N,N-dimethylaminododecyl (meth)acrylate, N-[3-(dimethylamino)propyl]methacrylamide, N-[3-(dimethylamino)propyl]acrylamide, N-[3-(dimethylamino)butyl]methacrylamide, N-[8-(dimethylamino)octyl]methacrylamide, N-[12-(dimethylamino)dodecyl]methacrylamide, N-[3-(diethylamino)propyl]methacrylamide, N-[3-(diethylamino)propyl]acrylamide;

maleic acid, fumaric acid, maleic anhydride and its half-esters, crotonic acid, itaconic acid, diallyldimethylammonium chloride, vinyl ethers (for example: methyl, ethyl, butyl or dodecyl vinyl ether), vinyl formamide, vinylmethylacetamide, vinylamine; methyl vinyl ketone, maleimide, vinylpyridine, vinylimidazole, vinylfuran, styrene, styrene sulfonate, allyl alcohol, and mixtures thereof.

Of these, particular preference is given to acrylic acid, methacrylic acid, maleic acid, fumaric acid, crotonic acid, maleic anhydride and its half-esters, methyl acrylate, methyl methacrylate, ethyl acrylate, ethyl methacrylate, n-butyl acrylate, n-butyl methacrylate, t-butyl acrylate, t-butyl methacrylate, isobutyl acrylate, isobutyl methacrylate, 2-ethylhexyl acrylate, stearyl acrylate, stearyl methacrylate, N-t-butylacrylamide, N-octylacrylamide, 2-hydroxyethyl acrylate, hydroxypropyl acrylate, 2-hydroxyethyl methacrylate, hydroxypropyl methacrylate, alkylene glycol (meth)acrylates, styrene, unsaturated sulfonic acids such as, for example, acrylamidopropane sulfonic acid, vinylpyrrolidone, vinylcaprolactam, vinyl ethers (e.g.: methyl, ethyl, butyl or dodecyl vinyl ether), vinylformamide, vinylmethylacetamide, vinylamine, 1-vinylimidazole, 1-vinyl-2-methylimidazole, N,N-dimethylaminomethyl methacrylate and N-[3-(dimethylamino)propyl]methacrylamide; 3-methyl-1-vinylimidazolium chloride, 3-methyl-1-vinylimidazolium methylsulfate, N,N-dimethylaminoethyl methacrylate, N-[3-(dimethylamino)propyl]methacrylamide quaternized with methyl chloride, methyl sulfate or diethyl sulfate.

Monomers having one basic nitrogen atom can be quaternized in the following manner:

Suitable for quaternizing the amines are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Other suitable quaternizing agents are dialkyl sulfates, in particular dimethyl sulfate or diethyl sulfate. The quaternization of the basic amines can also be carried out with alkylene oxides such as ethylene oxide or propylene oxide in the presence of acids. Preferred quaternizing agents are: methyl chloride, dimethyl sulfate or diethyl sulfate.

The quaternization can be carried out before the polymerization or after the polymerization.

In addition, it is possible to use the reaction products of unsaturated acids, such as, for example, acrylic acid or methacrylic acid, with a quaternized epichlorohydrin of the formula (VI) (R²⁶=C₁- to C₄₀-alkyl).

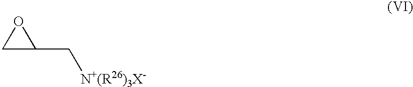

Examples thereof are: (meth)acryloyloxyhydroxypropyltrimethylammonium chloride and (meth)acryloyloxyhydroxypropyltriethylammonium chloride.

The basic monomers can also be cationized, by neutralizing them with mineral acids, such as, for example, sulfuric acid, hydrochloric acid, hydrobromic acid, hydroiodic acid, phosphoric acid or nitric acid, or with organic acids, such as, for example, formic acid, acetic acid, lactic acid, or citric acid.

In addition to the abovementioned comonomers, it is also possible to use, as comonomers (c), “macromonomers” such as, for example, silicone-containing macromonomers having one or more free-radically polymerizable groups or alkyloxazoline macromonomers, as described, for example, in EP 408 311.

Furthermore, it is possible to use monomers containing fluorine, as described, for example, in EP 558423, compounds which have a crosslinking action or compounds which regulate the molecular weight, in combination or alone.

Regulators which can be used are the customary compounds known to the person skilled in the art, such as, for example, sulfur compounds (e.g. mercaptoethanol, 2-ethylhexyl thioglycolate, thioglycolic acid or dodecyl mercaptan), and tribromochloromethane and other compounds which have a regulating effect on the molecular weight of the resulting polymers.

In some instances, it is also possible to use silicone compounds which contain thiol groups.

Preference is given to using silicone-free regulators.

Crosslinking monomers which can be used are compounds having at least two ethylenically unsaturated double bonds, such as, for example, esters of ethylenically unsaturated carboxylic acids, such as acrylic acid or methacrylic acid and polyhydric alcohols, ethers of at least dihydric alcohols such as, for example, vinyl ethers or allyl ethers.

Examples of the parent alcohols are dihydric alcohols such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, neopentyl glycol hydroxypivalate, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans having molecular weights of in each case 200 to 10 000. Apart from the homopolymers of ethylene oxide and propylene oxide, it is also possible to use block copolymers of ethylene oxide or propylene oxide or copolymers which contain ethylene oxide and propylene oxide groups in incorporated form. Examples of parent alcohols having more than two OH groups are trimethylolpropane, glycerol, pentaerythritol, 1,2,5-pentanetriol, 1,2,6-hexanetriol, triethoxycyanuric acid, sorbitan, sugars such as sucrose, glucose, mannose. It is of course also possible to use the polyhydric alcohols following reaction with ethylene oxide or propylene oxide, as the corresponding ethoxylates or propoxylates respectively. The polyhydric alcohols can also firstly be converted into the corresponding glycidyl ethers by reaction with epichlorohydrin.

Further suitable crosslinkers are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the above-described polyhydric alcohols, for example of oleic acid, crotonic acid, cinnamic acid or 10-undecenoic acid.

Also suitable are straight-chain or branched, linear or cyclic aliphatic or aromatic hydrocarbons which have at least two double bonds which, in the case of aliphatic hydrocarbons, must not be conjugated, e.g., divinylbenzene, divinyltoluene, 1,7-octadiene, 1,9-decadiene, 4-vinyl-1-cyclohexene, trivinylcyclohexane or polybutadienes having molecular weights of from 200 to 20 000.

Also suitable are amides of unsaturated carboxylic acids, such as, for example, acrylic acid and methacrylic acid, itaconic acid, maleic acid and N-allylamines of at least difunctional amines, such as, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophorone diamine. Also suitable are the amides of allylamine and unsaturated carboxylic acids such as acrylic acid, methacrylic acid, itaconic acid, maleic acid, or at least dibasic carboxylic acids as have been described above.

Further suitable crosslinkers are triallylamine or corresponding ammonium salts, e.g. triallylmethylammonium chloride or triallylmethylammonium methyl sulfate.

It is also possible to use the N-vinyl compounds of urea derivatives, at least difunctional amides, cyanurates or urethanes, for example of urea, ethyleneurea, propyleneurea or tartramide, e.g. N,N′-divinylethyleneurea or N,N′-divinylpropyleneurea.

Further suitable crosslinkers are divinyldioxane, tetraallylsilane or tetravinylsilane.

Particularly preferred crosslinkers are, for example, methylenebisacrylamide, divinylbenzene, triallylamine and triallylammonium salts, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin, and allyl or vinyl ethers of polyhydric alcohols, for example 1,2-ethanediol, 1,4-butanediol, diethylene glycol, trimethylolpropane, glycerol, pentaerythritol, sorbitan and sugars such as sucrose, glucose, mannose.

Very particularly preferred crosslinkers are pentaerythritol triallyl ethers, allyl ethers of sugars such as sucrose, glucose, mannose, divinylbenzene, methylenebisacrylamide, N,N′-divinylethyleneurea, and (meth)acrylic esters of glycol, butanediol, trimethylolpropane or glycerol or (meth)acrylic esters of glycol, butanediol, trimethylolpropane or glycerol reacted with ethylene oxide and/or epichlorohydrin.

The proportion of monomers which have a crosslinking action is 0 to 10% by weight, preferably 0 to 5% by weight, very particularly preferably 0 to 2% by weight.

In the polymerization for the preparation of the polymers according to the invention, in some instances other polymers, such as, for example, polyamides, polyurethanes, polyesters, homo- and copolymers of ethylenically unsaturated monomers, polyvinyl alcohol, may also be present. Examples of such polymers, some of which are also used in cosmetics, are the polymers known under the trade names Amerhold™, Ultrahold™, Luviquat™, Luviquat Care™, Ultrahold Strong™, Luviflex™ VBM, Luvimer™, Acronal™, Acudyne™, Stepanhold™, Lovocryl™, Versatyl™, Amphomer™, or Eastma AQ™.

The comonomers (c) according to the invention can, provided they contain ionizable groups, be partially or completely neutralized with acids or bases before or after the polymerization in order, for example, to adjust the solubility or dispersibility in water to a desired degree.

Neutralizing agents for monomers carrying acid groups which can be used are, for example, mineral bases such as sodium carbonate, alkali metal hydroxides and ammonia, organic bases such as aminoalcohols, specifically 2-amino-2-methyl-1-propanol, monoethanolamine, diethanolamine, triethanolamine, triisopropanolamine, tri[(2-hydroxy)1-propyl]amine, 2-amino-2-methyl-1,3-propanediol, 2-amino-2-hydroxymethyl-1,3-propanediol and diamines, such as, for example, lysine.

To prepare the polymers, the monomers of component a) can be polymerized in the presence of the polyethers either using initiators which form free radicals, or by the action of high-energy radiation, which is also intended to mean the action of high-energy electrons.

Initiators which can be used for the free-radical polymerization are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butylperoxy-2-ethyl hexanoate, tert-butyl permaleate, cumene hydroperoxide, diisopropyl peroxydicarbamate, bis-(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, azobis-(2-amidinopropane) dihydrochloride or 2,2′-azobis(2-methyl-butyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate.

Preference is given to using organic peroxides.

The amounts of initiator or initiator mixtures used, based on monomer used, are between 0.01 and 10% by weight, preferably between 0.1 and 5% by weight.

The polymerization is carried out in the temperature range from to 200° C., preferably in the range from 50 to 140° C., particularly preferably in the range from 60 to 110° C. It is usually carried out under atmospheric pressure, but can also be carried out under reduced or increased pressure, preferably between 1 and 5 bar.

The polymerization can, for example, be carried out as solution polymerization, bulk polymerization, emulsion polymerization, inverse emulsion polymerization, suspension polymerization, inverse suspension polymerization or precipitation polymerization, without the possible methods being limited thereto.

In the case of bulk polymerization, the procedure may involve dissolving the polyether-containing compound b) in at least one monomer of group a) and possibly other comonomers of group c) and, after the addition of a polymerization initiator, fully polymerizing the mixture. The polymerization can also be carried out semicontinuously by firstly introducing some, e.g. 10%, of the mixture to be polymerized comprising the polyether-containing compound b), at least one monomer from group a), possibly other comonomers of group c) and initiator, heating the mixture to the polymerization temperature and, after the polymerization has started, adding the remainder of the mixture to be polymerized in accordance with the progress of the polymerization. The polymers can also be obtained by initially introducing the polyether-containing compounds of group b) into a reactor, heating them to the polymerization temperature and adding at least one monomer of group a), possibly other comonomers of group c) and polymerization initiator either in one portion, step by step or, preferably, continuously, and polymerizing.

If desired, the above-described polymerization can also be carried out in a solvent. Suitable solvents are, for example, alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of dihydric alcohols, diethylene glycol, triethylene glycol, glycerol and dioxane. The polymerization can also be carried out in water as solvent. In this case, the initial charge is a solution which, depending on the amount of monomers of component a) added, is soluble in water to a greater or lesser degree. In order to convert water-insoluble products, which can form during the polymerization, into solution, it is possible, for example, to add organic solvents, such as monohydric alcohols having from 1 to 3 carbon atoms, acetone or dimethylformamide. However, in the case of polymerization in water, it is also possible to convert the water-insoluble polymers into a finely divided dispersion by addition of customary emulsifiers or protective colloids, e.g. polyvinyl alcohol.

The emulsifiers used are, for example, ionic or nonionic surfactants whose HLB value is in the range from 3 to 13. The definition of the HLB value can be found in the publication by W. C. Griffin, J. Soc. Cosmetic Chem., Volume 5, 249 (1954).

The amount of surfactants, based on the polymer, is 0.1 to 10% by weight. Using water as solvent gives solutions or dispersions of the polymers. If solutions of the polymer are prepared in an organic solvent or in mixtures of an organic solvent and water, then, per 100 parts by weight of the polymer, 5 to 2 000, preferably 10 to 500, parts by weight of the organic solvent or of the solvent mixture are used.

Preference is given to polymers obtainable by free-radical polymerization of

| | | |
|---|---|---|
| a) | 10-98% | by weight of at least one vinyl ester of C₁—C₂₄ |
| | | carboxylic acids in the presence of |
| b) | 2-90% | by weight of at least one polyether-containing |
| | | compound and |
| c) | 0-50% | by weight of one or more further copolymerizable |
| | | monomers. |

Particular preference is given to polymers obtainable by free-radical polymerization of

| | | |
|---|---|---|
| a) | 50-97% | by weight of at least one vinyl ester of C₁-C₂₄- |
| | | carboxylic acids in the presence of |
| b) | 3-50% | by weight of at least one polyether-containing |
| | | compound and |
| c) | 0-30% | by weight of one or more further copolymerizable |
| | | monomers. |

Very particular preference is given to polymers obtainable by free-radical polymerization of

| | | |
|---|---|---|
| a) | 65-97% | by weight of at least one vinyl ester of |
| | | C₁-C₂₄-carboxylic acids in the presence of |
| b) | 3-40% | by weight of at least one polyether-containing |
| | | compound and |
| c) | 0-20% | by weight of one or more further copolymerizable |
| | | monomers. |

To prepare the polymers used according to the invention, the ester groups of the original monomers a) and optionally of other monomers are cleaved after the polymerization by hydrolysis, alcoholysis, or aminolysis. This process step is generally referred to below as hydrolysis. The hydrolysis takes place in a manner known per se by the addition of a base, preferably the addition of a sodium or potassium hydroxide solution in water and/or alcohol. Particular preference is given to using methanolic sodium or potassium hydroxide solutions. The hydrolysis is carried out at temperatures in the range from 10 to 80° C., preferably in the range from 20 to 60° C. The degree of hydrolysis depends on the amount of base used, on the hydrolysis temperature, on the hydrolysis time and the water content of the solution.

The degree of hydrolysis of the polyvinyl ester groups can be up to 100%, preferably in the range from 40 to 100%, particularly preferably in the range from 65 to 100%, very particularly preferably in the range from 80 to 100%.

The polymers prepared in this way can then be cationized by reaction of hydroxyl and/or amino functions present in the polymer with epoxides of the formula VI (R²⁶=C₁- to C₄₀-alkyl).

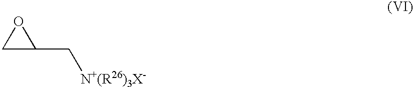

For this, the hydroxyl groups of the polyvinyl alcohol units and vinylamine units, formed by hydrolysis of vinylformamide, can preferably be reacted with the epoxides.

The epoxides of the formula VI can also be produced in situ by reaction of the corresponding chlorohydrins with bases, for example sodium hydroxide.

Preference is given to using 2,3-epoxypropyltrimethylammonium chloride or 3-chloro-2-hydroxypropyltrimethylammonium chloride.

The K values of the polymers should be in the range from 10 to 300, preferably 25 to 250, particularly preferably 25 to 200, very particularly preferably in the range from 30 to 150. The K value desired in each case can be adjusted in a manner known per se through the composition of the feed substances. The K values are determined in accordance with Fikentscher, Cellulosechemie, Vol. 13, pp. 58 to 64, and 71 to 74 (1932) in N-methylpyrrolidone at 25° C. and polymer concentrations which, depending on the K value range, are between 0.1% by weight and 5% by weight.

The polymer solutions can be steam distilled to remove any solvents. After the steam distillation, aqueous solutions or dispersions are obtained depending on the degree of hydrolysis, type of polyethers b), of vinyl esters a) and any monomers c) used.

The polymers obtained can also be subsequently crosslinked by reacting the hydroxyl groups or amino groups in the polymer with at least bifunctional reagents. In the case of low degrees of crosslinking, water-soluble products are obtained, and in the case of high degrees of crosslinking, water-swellable or insoluble products are obtained.

For example, the polymers according to the invention can be reacted with dialdehydes and diketones, e.g. glyoxal, glutaraldehyde, succindialdehyde or terephthalaldehyde. Also suitable are aliphatic or aromatic carboxylic acids, for example maleic acid, oxalic acid, malonic acid, succinic acid or citric acid, or carboxylic acid derivatives, such as carboxylic esters, anhydrides or halides. Also suitable are polyfunctional epoxides, e.g. epichlorohydrin, glycidyl methacrylate, ethylene glycol diglycidyl ether, 1,4-butanediol diglycidyl ether or 1,4-bis(glycidyloxy)benzene. Also suitable are diisocyanates, for example hexamethylene diisocyanate, isophorone diisocyanate, methylenediphenyl diisocyanate, toluylene diisocyanate or divinylsulfone.

Also suitable are inorganic compounds, such as boric acid or boric acid salts, for example sodium metaborate, borax (disodium tetraborate), and salts of polyvalent cations, e.g. copper(II) salts, such as copper(II) acetate or zinc, aluminum, titanium salts.

Boric acid and/or boric acid salts, such as sodium metaborate or disoium tetraborate, are preferably suitable for the subsequent crosslinking. In this connection, boric acid and/or boric acid salts can, preferably as salt solutions, be added to the solutions of the polymers according to the invention. Preference is given to adding boric acid and/or boric acid salts to the aqueous polymer solutions.

The boric acid and/or boric acid salts can be added to the polymer solutions directly after preparation. It is, however, also possible to add the boric acid and/or boric acid salts subsequently to the cosmetic formulations containing the polymers according to the invention, or to add them during the preparation process of the cosmetic formulations.

The proportion of boric acid and/or boric acid salts, based on the polymers according to the invention, is 0 to 15% by weight, preferably 0 to 10% by weight, particularly preferably 0 to 5% by weight.

The polymer solutions and dispersions can be converted into powder form by a variety of drying methods, such as, for example, spray drying, fluidized spray drying, drum drying or freeze drying. The drying method used in preference is spray drying. The dry polymer powder obtained in this way can be used to prepare an aqueous solution or dispersion again, by dissolution or redispersion in water. Conversion into powder form has the advantage of better storability, easier transportation, and a lower propensity for microbial attack.

Instead of the steam distilled polymer solutions, the alcoholic polymer solutions can also be directly converted into powder form.

The polymers according to the invention are highly suitable for use in skin cosmetic formulations, in particular for the preparation of skin care compositions, such as face-, eye- and footcare compositions, body lotions, shower gels, sun protection compositions and compositions for decorative skin cosmetics, and for the preparation of skincare compositions as day and night creams, vitamin creams, bleaching creams, W/O or O/W skin creams, peeling creams, lipsticks, foundation, face masks, peel-off masks and mascara.

In addition, the polymers according to the invention are suitable for use in dental and oral care compositions, such as mouthwashes and toothpaste, and as denture adhesives.

The copolymers according to the invention are present in the skin care compositions in an amount of from about 0.001 to 25% by weight, preferably 0.05 to 20% by weight, based on the total weight of the composition.

Depending on the field of application, the compositions according to the invention can be applied in a form suitable for skin care, such as, for example, as cream, foam, gel, stick, powder, spray or mousse.

The skin cosmetic formulations according to the invention comprise, in a preferred embodiment,

| | | |
|---|---|---|
| a) | 0.05-20% | by weight of the polymer according to the |
| | | invention, prepared by free-radical |
| | | polymerization of vinyl esters and |
| | | optionally further polymerizable monomers in |
| | | the presence of polyether-containing |
| | | compounds |
| b) | 20-99.95% | by weight of water and/or solvents and/or an |
| | | oil component |
| c) | 0-79.5% | by weight of further constituents. |

Suitable solvents which are to be mentioned in particular are lower monoalcohols or polyols having 1 to 6 carbon atoms or mixtures thereof; preferred monoalcohols or polyols are ethanol, i-propanol, n-propanol, propylene glycol, glycerol and sorbitol.

Suitable fatty substances which may be present are mineral, animal, vegetable or synthetic oils or waxes, fatty acids, fatty acid esters, such as triglycerides of C₆-C₁₈-fatty acids, fatty alcohols, vaseline, paraffin, lanolin, hydrogenated lanolin, acetylated lanolin and silicone oil.

Further constituents are understood as meaning the additives customary in cosmetics, for example propellants, antifoams, interface-active compounds, i.e. surfactants, emulsifiers, foam formers and solubilizers. The interface-active compounds used can be anionic, cationic, amphoteric or neutral. Further customary constituents can also be, for example, preservatives, perfume oils, opacifiers, light protection agents, UV filters, cosmetic active ingredients, such as phytantriol, vitamin A, E and C, retinol, bisabolol, panthenol, collagen, alpha- and beta-hydroxycarboxylic acids, protein hydrolyzates, bleaches, tanning agents (e.g. dihydroxyacetone), stabilizers, pH regulators, colorants, viscosity regulators, gel formers, salts, moisturizers, refatting agents and further customary additives.

These also include all styling and conditioning polymers known in cosmetics which can be used in combination with the polymers according to the invention, in cases where very specific properties are to be set.

Suitable traditional cosmetic polymers are, for example, anionic polymers. Such anionic polymers are homo- and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset® P.U.R.), polyureas and polyvinyl alcohols. Particularly suitable polymers are copolymers of t-butyl acrylate, ethyl acrylate, methacrylic acid (e.g. Luvimer® 100P), copolymers of N-tert-butylacrylamide, ethyl acrylate, acrylic acid (Ultrahold® 8, strong), copolymers of vinyl acetate, crotonic acid and optionally other vinyl esters (e.g. Luviset® grades), maleic anhydride copolymers, optionally reacted with alcohols, anionic polysiloxanes, e.g. carboxy-functional ones, copolymers of vinylpyrrolidone, t-butyl acrylate, methacrylic acid (e.g. Luviskol® VBM).

Very particularly preferred anionic polymers are acrylates with an acid number greater than or equal to 120 and copolymers of t-butyl acrylate, ethyl acrylate and methacrylic acid.

Other suitable skin cosmetic polymers are cationic polymers with the name polyquaternium according to INCI, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethylsulfate (Luviquat® PQ 11), copolymers of N-vinylcaprolactam-N-vinylpyrrolidone/N-vinylimidazolium salts (Luviquat® Hold); cationic cellulose derivatives (polyquaternium-4 and -10), acrylamide copolymers (polyquaternium-7).

Other suitable polymers are also neutral polymers such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethyleneimines and salts thereof, polyvinylamines and salts thereof, cellulose derivatives, polyaspartic acid salts and derivatives.

To establish certain properties, the preparations can also additionally comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and amino-functional silicone compounds such as amodimethicones (CTFA).

The copolymers according to the invention are used in cosmetic or dermatological preparations, and are prepared in accordance with the customary guidelines familiar to the person skilled in the art.

Such formulations are advantageously in the form of emulsions, preferably as water-in-oil (W/O) or oil-in-water (O/W) emulsions. It is, however, also possible according to the invention and in some instances advantageous to choose other types of formulation, for example hydrodispersions, gels, oils, oleogels, multiple emulsions, for example in the form of W/O/W or O/W/O emulsions, anhydrous ointments or ointment bases etc.

The preparation of emulsions which can be used according to the invention is carried out by known methods. The choice of additives specific to the type of emulsion and the preparation of suitable emulsions is described, for example, in Schrader, Grundlagen und Rezepturen der Kosmetika [Bases and Formulations in Cosmetics], Hüthig Buch Verlag, Heidelberg, 2nd edition, 1989, third part, to which reference is expressly made here.

In a preferred embodiment, emulsions according to the invention comprise

| | | |
|---|---|---|
| a) | 0.05-10% | by weight of the polymer according to the |
| | | invention, prepared by free-radical |
| | | polymerization of vinyl esters and |
| | | optionally further polymerizable monomers in |
| | | the presence of polyether-containing |
| | | compounds |
| b) | 10-94.94% | by weight of water |
| c) | 5-89.94% | by weight of an oil component |
| d) | 0.01-40% | by weight of an emulsifier |
| e) | 0-74.94% | by weight of further constituents. |

A preferred form of a skin cosmetic formulation of the polymers according to the invention is a W/O emulsion which comprises an aqueous phase which is emulsified in an oil or fatty phase using a suitable emulsifier system.

| | | |
|---|---|---|
| a) | 0.05-10% | by weight of the polymer according to the |
| | | invention, prepared by free-radical |
| | | polymerization of vinyl esters and |
| | | optionally further polymerizable monomers in |
| | | the presence of polyether-containing |
| | | compounds |
| b) | 20-77.95% | by weight of water |
| c) | 20-77.95% | by weight of an oil component |
| d) | 2-35% | by weight of an emulsifier |
| e) | 0-55.95% | by weight of further constituents. |

The emulsifiers are those which are customarily used in this type of emulsion. They are, for example, chosen from: C₁₂-C₁₈-sorbitan fatty acid esters; esters of hydroxystearic acid and C₁₂-C₃₀-fatty alcohols; mono- and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols having a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium, calcium, lithium, zinc or aluminum lanolate and hydrogenated lanolin or lanolin alcohol.

Suitable fatty components which may be present in the fatty phase of the emulsions include: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start point at atmospheric pressure is at about 250° C. and whose distillation end point is at 410° C., such as, for example, vaseline oil; esters of saturated or unsaturated fatty acids, such as alkyl myristates, e.g. isopropyl, butyl or cetyl myristate, hexadecyl stearate, ethyl or isopropyl palmitate, octanoic or decanoic acid triglycerides and cetyl ricinoleate.

The fatty phase can also comprise silicone oils which are soluble in other oils, such as dimethylpolysiloxane, methylphenylpolysiloxane and the silicone glycol copolymer, fatty acids and fatty alcohols.

In order to promote the retention of oils, it is also possible to use waxes, such as, for example, carnauba wax, candellila wax, beeswax, microcrystalline wax, ozokerite wax and Ca, Mg and Al oleates, myristates, linoleates and stearates.

In general, these water-in-oil emulsions are prepared by adding the fatty phase and the emulsifier to the charging container. These are then heated at a temperature of from 70 to 75° C., then the oil-soluble ingredients are added and, with stirring, water is added which has been heated beforehand to the same temperature and in which the water-soluble ingredients have been dissolved beforehand; the mixture is stirred until an emulsion of the desired fineness is obtained, which is then left to cool to room temperature, if necessary with gentle stirring.

In addition, a care emulsion according to the invention can be in the form of a O/W emulsion. An emulsion of this type usually comprises an oil phase, emulsifiers which stabilize the oil phase in the water phase, and an aqueous phase which, where appropriate, is in thickened form.

| | | |
|---|---|---|
| a) | 0.05-10% | by weight of the polymer according to the |
| | | invention, prepared by free-radical |
| | | polymerization of vinyl esters and |
| | | optionally further polymerizable monomers in |
| | | the presence of polyether-containing |
| | | compounds |
| b) | 40-96.95% | by weight of water |
| c) | 1-44.95% | by weight of an oil component |
| d) | 1-35% | by weight of an emulsifier |
| e) | 0-10% | by weight of a gel former |
| f) | 0-57.95% | by weight of further constituents. |

Suitable emulsifiers are preferably O/W emulsifiers. Suitable emulsifiers may be nonionic, cationic or anionic.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, quaternium-1 to x (INCI).

Anionic emulsifiers can, for example, be chosen from the group of alkylsulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, the alkyl ether phosphates and alkyl ether carboxylates can have between 1 and 10 ethylene oxide or propylene oxide units, preferably 1 to 3 ethylene oxide units, in the molecule.

Suitable oil components which may be present in the fatty phase of the emulsions include: hydrocarbon oils, such as paraffin oil, purcellin oil, perhydrosqualene and solutions of microcrystalline waxes in these oils; esters of saturated and/or unsaturated, branched and/or unbranched C₃-C₃₀-alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example isopropyl stearate, hexyldecyl stearate, oleyl oleate, isopropyl, butyl or cetyl myristate, ethyl or isopropyl palmitate, octanoic or decanoic acid triglycerides and cetyl ricinoleate; and also synthetic, semisynthetic and natural mixtures of such esters, such as jojoba oil, animal or vegetable oils, such as sweet almond oil, avocado oil, calophylum oil, lanolin and derivatives thereof, castor oil, sesame oil, palm oil, almond oil, olive oil, jojoba oil, karite oil, hoplostethus oil; mineral oils whose distillation start point at atmospheric pressure is at about 250° C. and whose distillation end point is at 410° C., such as, for example, vaseline oil. The fatty phase can also comprise silicone oils which are soluble in other oils, such as dimethylpolysiloxane, cyclomethicone, methylphenylpolysiloxane and the silicone glycol copolymer, octamethylcyclotetrasiloxane.

In order to promote the retention of oils, it is also possible to use waxes, such as, for example, carnauba wax, candellila wax, beeswax, microcrystalline wax, ozokerite wax and Ca, Mg and Al oleates, myristates, linoleates and stearates.

Customary thickeners or gel formers which can be used are, for example, crosslinked polyacrylic acids and derivatives thereof, polysaccharides such as xanthan gum, or hydroxycarboxymethylcellulose, fatty alcohols.

The gel formers which can be used are any gel formers customary in cosmetics. These include slightly crosslinked polyacrylic acid, for example carbomer (INCI), cellulose derivatives, e.g. hydroxypropylcellulose, hydroxyethylcellulose, carboxymethylcellulose, cationically modified celluloses, polysaccharides, e.g. xanthum gum or alginates, caprylic/capric triglycerides, sodium acrylates copolymer, polyquaternium-32 (and) paraffinum liquidum (INCI), sodium acrylates copolymer (and) paraffinum liquidum (and) PPG-1 trideceth-6, acrylamidopropyl trimonium chloride/acrylamide copolymer, steareth-10 allyl ether acrylates copolymer, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44, polyvinyl alcohol and polyvinylpyrrolidone.

The preparation can be carried out by melting the oil phase at 80° C.; the water-soluble constituents are dissolved in hot water, and the melted oil phase is slowly added with stirring; the mixture is then homogenized and stirred until cold.

The copolymers according to the invention are also suitable for use in washing and shower gel formulations and also bath preparations.

In addition to the polymers according to the invention, such formulations usually comprise anionic surfactants as base surfactants, and amphoteric and nonionic surfactants as cosurfactants, and lipids, perfume oils, dyes, organic acids, preservatives and antioxidants, and thickeners/gel formers, skin-conditioning agents and humectants.

The formulations comprise 2 to 50% by weight of surfactants, preferably 5 to 40% by weight, particularly preferbaly 8 to 30% by weight.

In the washing and shower gel formulations and bath preparations it is possible to use all anionic, neutral, amphoteric or cationic surfactants customarily used for skin cleansing.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium and ammonium and triethanolamine salts. The alkyl ether sulfates, alkyl ether phosphates and alkyl ether carboxylates can have between 1 and 10 ethylene oxide or propylene: oxide units, preferably 1 to 3 ethylene oxide units, in the molecule.

Suitable examples are sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkylglycinates, alkyl carboxyglycinates, alkyl amphoacetates or -propionates, alkyl amphodiacetates or -dipropionates.

It is possible, for example, to use cocodimethylsulfopropylbetaine, laurylbetaine, cocamidopropylbetaine or sodium cocamphopropionate.

Examples of suitable nonionic surfactants are the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkyl alkanolamides, fatty acid esters of polyethylene glycols, ethoxylated fatty acid amides, alkyl polyglycosides or sorbitan ether esters.

In addition, the washing and shower gel formulations and bath preparations may comprise customary cationic surfactants, such as, for example, quaternary ammonium compounds, for example cetyltrimethylammonium chloride.

In addition, further customary cationic polymers can also be used, such as, for example, copolymers of acrylamide and dimethyldiallylammonium chloride (polyquaternium-7), cationic cellulose derivatives (polyquaternium-10), guar hydroxypropyltrimethylammonium chloride (INCI: hydroxypropyl guar hydroxypropyltrimonium chloride), copolymers of N-vinylpyrrolidone and quaternized N-vinylimidazole (polyquaterinium-16, -44, -46) and others.

In addition, the washing and shower gel formulations and bath preparations can comprise thickeners, such as, for example, common salt, PEG-55, propylene glycol oleate, PEG-120 methyl glucose dioleate and others, and preservatives, further active ingredients and auxiliaries and water.

### PREPARATION EXAMPLES

Preparation Procedure for Examples 1 to 32

The polyether-containing compound is heated to 80° C. in a polymerization vessel with stirring and under a gentle stream of nitrogen. With stirring, vinyl acetate and optionally the other monomers are metered in over the course of 3 h. At the same time, a solution of 1.4 g of tert-butyl perpivalate in 30 g of methanol is likewise added over 3 h. The mixture is then stirred for a further 2 h at 80° C. After cooling, the polymer is dissolved in 450 ml of methanol. For hydrolysis, 50 ml of a 10% strength methanolic sodium hydroxide solution are added at 30° C. After about 40 min, the reaction is terminated by addition of 750 ml of 1% strength acetic acid. The methanol is removed by distillation.

The K values were determined at a concentration of 1% in N-methylpyrrolidone.

TABLE

| | | | | | Degree of |
|---|---|---|---|---|---|
| | | | | K | hydrolysis |
| Example | Graft base | Vinyl ester | Comonomer | value | [%] |
| 1 | PEG 1500¹ | Vinyl | — | 47 | >95 |
| | 72 g | acetate, | | | |
| | | 410 g | | | |
| 2 | PEG 4000 | Vinyl | — | 51 | >95 |
| | 72 g | acetate, | | | |
| | | 410 g | | | |
| 3 | PEG 6000, | Vinyl | — | 54 | >95 |
| | 72 g | acetate, | | | |
| | | 410 g | | | |
| 4 | PEG 6000, | Vinyl | — | 49 | >95 |
| | 137 g | acetate, | | | |
| | | 410 g | | | |
| 5 | PEG 6000, | Vinyl | — | 73 | >95 |
| | 22 g | acetate | | | |
| | | 410 g | | | |
| 6 | PEG 6000, | Vinyl | | 42 | >95 |
| | 410 g | acetate | | | |
| | | 410 g | | | |
| 7 | PEG 9000, | Vinyl | — | 58 | >95 |
| | 137 g | acetate, | | | |
| | | 410 g | | | |
| 8 | Polyglycerol 2200, | Vinyl | — | 66 | >95 |
| | 72 g | acetate, | | | |
| | | 410 g | | | |
| 9 | PEG-PPG block | Vinyl | — | 45 | >95 |
| | copolymer 8000², | acetate, | | | |
| | 72 g | 410 g | | | |
| 10 | Methyl polyethylene | Vinyl | — | 47 | >95 |
| | glycol 2000³ | acetate, | | | |
| | 72 g | 410 g | | | |
| 11 | Alkyl polyethylene glycol | Vinyl | — | 48 | >95 |
| | 3500⁴ | acetate, | | | |
| | 72 g | 410 g | | | |
| 12 | PPG 4000⁵ | Vinyl | | 50 | >95 |
| | 72 g | acetate | | | |
| | | 410 g | | | |
| 13 | PEG 20000 | Vinyl | — | 69 | >95 |
| | 72 g | acetate, | | | |
| | | 410 g | | | |
| 14 | PEG 20000 | Vinyl | — | 64 | >95 |
| | 103 g | acetate, | | | |
| | | 410 g | | | |
| 15 | PEG 20000 | Vinyl | — | 59 | >95 |
| | 137 g | acetate, | | | |
| | | 410 g | | | |
| 16 | PEG 20000 | Vinyl | — | 55 | >86 |
| | 615 g | acetate, | | | |
| | | 410 g | | | |
| 17 | PEG 35000 | Vinyl | — | 77 | >95 |
| | 72 g | acetate, | | | |
| | | 410 g | | | |
| 18 | PEG 35000 | Vinyl | — | 80 | >95 |
| | 137 g | acetate, | | | |
| | | 410 g | | | |
| 19 | PEG 35000 | Vinyl | — | 65 | 97 |
| | 205 g | acetate, | | | |
| | | 410 g | | | |
| 20 | Dimethicone copolyol⁶ | Vinyl | — | 58 | >95 |
| | 202 g | acetate, | | | |
| | | 410 g | | | |
| 21 | Poly(sodium | Vinyl | | 43 | >95 |
| | methacrylate-co- | acetate, | | | |
| | methylpolyethylene glycol | 410 g | | | |
| | methacrylate)⁷ | | | | |
| | 103 g, | | | | |
| 22 | ethoxylated | Vinyl | | 52 | >95 |
| | polyethyleneimine⁸ | acetate, | | | |
| | 137 g | 410 g | | | |
| 23 | PEG 6000, | Vinyl | Methyl methacrylate, | 47 | >95 |
| | 72 g | acetate, | 24 g | | |
| | | 386 g | | | |
| 24 | PEG 20000, | Vinyl | N-Vinylpyrrolidone, | 61 | >95 |
| | 72 g | acetate, | 82 | | |
| | | 328 g | | | |
| 25 | PEG 20000, | Vinyl | 3-Methyl-1- | 53 | >95 |
| | 72 g | acetate, | vinylimidazolium methyl | | |
| | | 362 g | sulfate, | | |
| | | | 48 g | | |
| 26 | PEG 6000, | Vinyl | N-Vinylformamide, | 57 | >95 |
| | 72 g | acetate, | 41 g | | |
| | | 367 g | | | |
| 27 | PEG 6000, | Vinyl | N-Vinylformamide, | 67 | >95 |
| | 72 g | acetate, | 82 g | | |
| | | 326 g | | | |
| 28 | PEG 35000, | Vinyl | | 59 | 96 |
| | 270 g | acetate, | | | |
| | | 410 g | | | |
| 29 | PEG 35000, | Vinyl | Pentaerythritol triallyl ether, | 71 | 95 |
| | 270 g | 410 g | 1.6 g | | |
| 30 | PEG 35000, | Vinyl | Pentaerythritol triallyl ether, | 65 | 94 |
| | 270 g | acetate, | 0.8 g | | |
| | | 410 g | | | |
| 31 | PEG 35000, | Vinyl | N,N‘-Divinyl-ethyleneurea | 73 | 95 |
| | 270 g | acetate, | 0.7 g | | |
| | | 410 g | | | |
| 32 | PEG 12000, | Vinyl | Pentaerythritol triallyl ether, | 50 | 94 |
| | 270 g | acetate, | 1.6 g | | |
| | | 410 g | | | |

¹PEG x: polyethylene glycol with an average molecular weight x
²Lutrol F 68 from BASF Aktiengesellschaft (PPG: polypropylene glycol)
³Pluriol A 2000 E from BASF Aktiengesellschaft
⁴Lutensol AT 80 from BASF Aktiengesellschaft (C₁₆-C₁₈ fatty alcohol + 80 EO)
⁵Polypropylene glycol with an average molecular weight of 4 000
⁶Belsil DMC 6031TM from Wacker Chemie GmbH
⁷Molar ratio of sodium methacrylate/methylpolyethylene glycol methacrylate 4:1, methylpolyethylene glycol with a molar mass of about 1 000
⁸Prepared from 12.5% of polyethyleneimine (average molecular weight 1 400) and 87.5% of ethylene oxide

### Example 33

### Reaction with 3-chloro-2-hydroxypropyltrimethylammonium Chloride

22 g of a 60% strength aqueous solution of 3-chloro-2-hydroxypropyltrimethylammonium chloride and 3.5 g of sodium hydroxide are added to 400 g of a 32.9% strength solution from example 3. The mixture is stirred for 3 hours at 60° C. and then for a further two hours at 90° C., giving a clear solution.

### Example 34

### Reaction with 3-chloro-2-hydroxypropyltrimethylammonium Chloride

46 g of a 60% strength aqueous solution of 3-chloro-2-hydroxypropyltrimethylammonium chloride and 6 g of sodium hydroxide are added to 400 g of a 15.3% strength solution from example 26. The mixture is stirred for 3 hours at 60° C. and then for a further two hours at 90° C., giving a clear solution.

### Example 35

At room temperature and with stirring, a 5% strength aqueous solution of disodium tetraborate (borax) is added to a 19.3% strength aqueous solution of the polymer from example 28 over the course of half an hour. An increase in the viscosity is observed.

| Amount of 5% strength borax | Brookfield viscosity (LVF, |
|---|---|
| solution added [g] | spindle 2, 30 rpm, 23° C.) [mPas] |
| 0 | 110 |
| 14.9 | 128 |
| 18.0 | 216 |
| 21.0 | 534 |
| 24.0 | 2228 |
| 26.9 | 7520¹ |
| 29.8 | 29190² |

1 Spindle 4, 30 rpm
2 Spindle 4, 6 rpm

### Unhydrolyzed Examples

### Examples 36 to 39

The polyether-containing compound is heated to 80° C. in a polymerization vessel with stirring and under a gentle stream of nitrogen. With stirring, vinyl acetate and optionally the other monomers are metered in over the course of 3 h. At the same time, a solution of 1.4 g of tert-butyl perpivalate in 30 g of methanol is likewise added over 3 h. The mixture is then stirred for a further 2 h at 80° C. After cooling, the polymer is dissolved in 450 ml of methanol and steam-distilled, giving an aqueous solution or dispersion.

| | Graft base | Vinyl ester | Comonomer | K value |
|---|---|---|---|---|
| 36 | PEG 1500¹ | Vinyl acetate, | — | 34 |
| | 410 g | 410 g | | |
| 37 | PEG 4000 | Vinyl acetate, | — | 45 |
| | 137 g | 410 g | | |
| 38 | PEG 6000, | Vinyl acetate, | — | 36 |
| | 205 g | 410 g | | |
| 39 | PEG 9000, | Vinyl acetate, | | 45 |
| | 410 g | 410 g | | |

### B Application Examples

### Application Example 1

### Skin Cream

A water/oil cream emulsion (skin cream A) according to the invention was firstly prepared in accordance with the following formulation:

| | | % by |
|---|---|---|
| | Additive | wt. |
| Cremophor A 6 | Ceteareth-6 and stearyl alcohol | 2.0 |
| Cremophor A 25 | Ceteareth-25 | 2.0 |
| Lanette O | Cetearyl alcohol | 2.0 |
| Imwitor 960 K | Glyceryl stearate SE | 3.0 |
| Paraffin oil | | 5.0 |
| Jojoba oil | | 4.0 |
| Luvitol EHO | Cetearyl octanoate | 3.0 |
| ABIL 350 | Dimethicone | 1.0 |
| Amerchol L 101 | Mineral oil and lanolin alcohol | 3.0 |
| Veegum Ultra | Magnesium aluminum silicate | 0.5 |
| 1,2-propylene glycol | Propylene glycol | 5.0 |
| Abiol | Imidazolindinylurea | 0.3 |
| Phenoxyethanol | | 0.5 |
| D-Panthenol USP | | 1.0 |
| Polymer (preparation | | 0.5 |
| example 28) | | |
| Water | | ad 100 |

Two comparison creams were prepared in the same way:

Skin Cream B (Without the Addition of Polymer)

The following comparison tests 1 and 2 were carried out with these skin creams A and B to assess the feel on the skin.

100 μl of the emulsion were distributed uniformly on the backs of the hands, and the feel of the skin was tested subjectively after a contact time of 30 minutes. In each case, two emulsions (right/left hand) were compared with one another. The test was carried out by 10 subjects in each case.

Grade Scale:

2 (significantly softer than comparison cream)

1 (somewhat softer than comparison cream)

0 (identical)

−1 (somewhat rougher than comparison cream)

−2 (significantly rougher than comparison cream)

Result of Comparison Test 1 (Comparison of Skin Cream A and Comparison Cream B):

| Grade | Number of subjects |
|---|---|
| 2 | 5 |
| 1 | 4 |
| 0 | 1 |
| −1 | — |
| −2 | — |

### Application Example 2

### Shower Gel

A shower gel formulation (shower gel A) of the invention was firstly prepared according to the following formulation:

| | Additive | % by wt. |
|---|---|---|
| Texapon NSO | Sodium laureth sulfate | 40.0 |
| Tego Betaine L7 | Cocamidopropylbetaine | 5.0 |
| Plantacare 2000 | Decyl glucoside | 5.0 |
| Perfume | | 0.2 |
| Polymer according to | | 0.2 |
| preparation example 25 | | |
| Euxyl K 100 | Benzyl alcohol, | 0.1 |
| | methylchloroisothiazolinone, | |
| | methylisothiazolinone | |
| D-Panthenol USP | | 0.5 |
| Citric acid (pH 6-7) | | q.s. |
| NaCl | | 2.0 |
| Water | | ad 100 |

Three comparison shower gels were prepared in the same way:

Shower Gel B: (Copolymer According to the Invention Replaced by the Same Amount of Cationically Modified Hydroxyethylcellulose)

Shower Gel C: (Without the Addition of Polymer)

The following comparison test 3 was carried out with shower gels A, B and C to determine the creaminess of the lather:

2.0 g of each of the abovementioned formulations were applied to the palm of the left hand, lathered with tapwater and, after rubbing for 1 minute between both hands, the feel of the lather in the palms of the hands was assessed:

Grade 1: very creamy

Grade 2: creamy

Grade 3: flat/lacking substance

Result of Comparison Test 3 (Average Grading from 10 Subjects):

| Shower gel | Average from 10 subjects |
|---|---|
| A | 1.3 |
| B | 2.1 |
| C | 2.8 |

### Application Example 3

### Humectant Formulation

Formulation A

| | | Additive | % by wt. |
|---|---|---|---|
| a) | Cremophor A6 | Ceteareth-6 and stearyl | 2.0 |
| | | alcohol | |
| | Cremophor A25 | Ceteareth-25 | 2.0 |
| | Paraffin oil | | |
| | (high viscosity) | | 10 |
| | Lanette O | Cetearyl alcohol | 2.0 |
| | Stearic acid | | 3.0 |
| | Nip-Nip | Methylparaben/propylparaben | 0.5 |
| | | 70:30 | |
| | Abiol | Imidazoldinylurea | 0.5 |
| b) | Polymer | | 3.0 |
| | (preparation example 3) | | |
| | Water | | ad 100.0 |

The two phases were heated to 80° C., phase a) was stirred into b), homogenized and stirred until cold, and then the mixture was adjusted to pH 6 with 10% strength aqueous NaOH solution.

A comparison cream (formulation B) was prepared in the same way without the addition of polymer.

A subject test on 8 subjects was carried out with formulations A and B. For this, the formulations were in each case applied to he forearm of the subjects in an amount of 2 mg/cm². After 30 min, the moisture content of the skin was determined using a Corneometer CM 825 (Khazaka & Courage). Following the application of formulation A, an average value of 45 corneometer units was measured, and with formulation B an average value of 35 was measured.

### Application Example 4

### O/W Cream for Retaining Skin Moisture

| | % by |
|---|---|
| Additive | wt. |
| Glycerol monostearate | 2.0 |
| Cetyl alcohol | 3.0 |
| Paraffin oil, subliquidum | 15.0 |
| Vaseline | 3.0 |
| Caprylic/capric triglyceride | 4.0 |
| Octyldodecanol | 2.0 |
| Hydrogenated coconut fat | 2.0 |
| Cetyl phosphate | 0.4 |
| Polymer (preparation example 33) | 3.0 |
| Glycerol | 3.0 |
| Sodium hydroxide | q.s. |
| Perfume oil | q.s. |
| Preservative | q.s. |
| Water | ad 100 |

### Application Example 5

### O/W Lotion

| | % by |
|---|---|
| Additive | wt. |
| Stearic acid | 1.5 |
| Sorbitan monostearate | 1.0 |
| Sorbitan monooleate | 1.0 |
| Paraffin oil, subliquidum | 7.0 |
| Cetyl alcohol | 1.0 |
| Polydimethylsiloxane | 1.5 |
| Glycerol | 3.0 |
| Polymer (preparation example 19) | 0.5 |
| Perfume oil | q.s. |
| Preservative | q.s. |
| Water | ad 100 |

### Application Example 6

### W/O Cream

| | % by |
|---|---|
| Additive | wt. |
| PEG-7 hydrogenated castor oil | 4.0 |
| Wool wax alcohol | 1.5 |
| Beeswax | 3.0 |
| Triglyceride, liquid | 5.0 |
| Vaseline | 9.0 |
| Ozokerite | 4.0 |
| Paraffin oil, subliquidum | 4.0 |
| Glycerol | 2.0 |
| Polymer (preparation example 20) | 2.0 |
| Magnesium sulfate*7H₂O | 0.7 |
| Perfume oil | q.s. |
| Preservative | q.s. |
| Water | ad 100 |

### Application Example 7

### Skin Care Hydrogel

| Additive | % by wt. |
|---|---|
| Polymer (preparation example 29) | 3.0 |
| Sorbitol | 2.0 |
| Glycerol | 3.0 |
| Polyethylene glycol 400 | 5.0 |
| Ethanol | 1.0 |
| Perfume oil | q.s. |
| Preservative | q.s. |
| Water | ad 100 |

### Application Example 8

### Hydrodispersion Gel

| Additive | % by wt. |
|---|---|
| Polymer (preparation example 26) | 3.0 |
| Sorbitol | 2.0 |
| Glycerol | 3.0 |
| Polyethylene glycol 400 | 5.0 |
| Triglyceride, liquid | 2.0 |
| Ethanol | 1.0 |
| Perfume oil | q.s. |
| Preservative | q.s. |
| Water | ad 100 |

### Application Example 9

### Liquid Soap

| Additive | % by wt. |
|---|---|
| Coconut fatty acid, potassium salt | 15 |
| Potassium oleate | 3 |
| Glycerol | 5 |
| Polymer (preparation example 16) | 2 |
| Glycerol stearate | 1 |
| Ethylene glycol distearate | 2 |
| Specific additives, complexing agents, fragrances | q.s. |
| Water | ad 100 |

### Application Example 10

### Body Care Cream

| | Additive | % by wt. |
|---|---|---|
| Cremophor A6 | Ceteareth-6 and stearyl alcohol | 2.0 |
| Cremophor A25 | Ceteareth-25 | 2.0 |
| Grape (Vitis vinifera) | | 6.0 |
| seed oil | | |
| Glyceryl stearate SE | | 3.0 |
| Cetearyl alcohol | | 2.0 |
| Dimethicone | | 0.5 |
| Luvitol EHO | Cetearyl octanoate | 8.0 |
| Oxynex 2004 | Propylene glycol, BHT, ascorbyl | 0.1 |
| | palmitate, glyceryl stearate, | |
| | citric acid | |
| Preservative | | q.s. |
| 1,2-propylene | | 3.0 |
| glycol USP | | |
| Glycerol | | 2.0 |
| EDTA BD | | 0.1 |
| D-Panthenol USP | | 1.0 |
| Water | | ad 100 |
| Polymer (preparation | | 1.5 |
| example 7) | | |
| Tocopheryl acetate | | 0.5 |

The formulation had a pH of 6.8.

In the application examples below, all the amounts are in % by weight.

### Application Example 9

### Liquid Foundation

| A | |
|---|---|
| 1.70 | glyceryl stearate |
| 1.70 | cetyl alcohol |
| 1.70 | ceteareth-6 |
| 1.70 | ceteareth-25 |
| 5.20 | caprylic/capric triglyceride |
| 5.20 | mineral oil |
| B | |
| q.s. | preservative |
| 4.30 | propylene glycol |
| 2.50 | polymer according to preparation example 11 |
| 59.50 | dist. water |
| C | |
| q.s. | perfume oil |
| D | |
| 2.00 | iron oxides |
| 12.00 | titanium dioxide |

Preparation:

Heat phase A and phase B separately to 80° C. Then mix phase B into phase A using a stirrer. Cool to 40° C. and add phase C and phase D. Homogenize repeatedly.

### Application Example 10

### Oil-Free Foundation

| A | |
|---|---|
| 0.35 | veegum |
| 5.00 | butylene glycol |
| 0.15 | xanthan gum |
| B | |
| 53.0 | dist. water |
| q.s. | preservative |
| 0.2 | polysorbate-20 |
| 1.6 | tetrahydroxypropylethylenediamine |
| C | |
| 1.0 | silica |
| 2.0 | nylon-12 |
| 4.15 | mica |
| 6.0 | titanium dioxide |
| 1.85 | iron oxides |
| D | |
| 4.0 | stearic acid |
| 1.5 | glyceryl stearate |
| 7.0 | benzyl laurate |
| 5.0 | isoeicosane |
| q.s. | preservative |
| E | |
| 1.0 | dist. water |
| 0.5 | panthenol |
| 0.1 | imidazolidinylurea |
| 5.0 | polymer according to preparation example 19 |

Preparation:

Wet phase A with butylene glycol, add to phase B and mix thoroughly. Heat phase AB to 75° C. Pulverize phase C feed substances, add to phase AB and homogenize thoroughly. Mix the substances of phase D, heat to 80° C. and add to phase ABC. Mix for some time until the mixture is homogeneous. Transfer the mixture to a vessel fitted with a propeller mixer. Mix feed substances of phase E, add to phase ABCD and mix thoroughly.

### Application Example 11

### Eyeliner

| A | |
|---|---|
| 40.6 | dist. water |
| 0.2 | disodium EDTA |
| q.s. | preservative |
| B | |
| 0.6 | xanthan gum |
| 0.4 | veegum |
| 3.0 | butylene glycol |
| 0.2 | polysorbate-20 |
| C | |
| 15.0 | iron oxide/Al powder/silica (e.g. Sicopearl Fantastico |
| | Gold ™ from BASF) |
| D | |
| 10.0 | dist. water |
| 30.0 | polymer according to preparation example 39 |

Preparation:

Premix phase B. Mix phase B into phase A using a propeller mixer, allowing the thickener to swell. Wet phase C with phase D, add everything to phase AB and mix thoroughly.

### Application Example 12

### Shimmering Gel

| A | |
|---|---|
| 32.6 | dist. water |
| 0.1 | disodium EDTA |
| 25.0 | carbomer (2% strength aqueous solution) |
| 0.3 | preservative |
| B | |
| 0.5 | dist. water |
| 0.5 | triethanolamine |
| C | |
| 10.0 | dist. water |
| 9.0 | polymer according to preparation example 31 |
| 1.0 | polyquaternium-46 |
| 5.0 | iron oxide |
| D | |
| 15.0 | dist. water |
| 1.0 | D-panthenol 50 P (panthenol and propylene glycol) |

Preparation:

Thoroughly mix the feed substances of phase A in the order given using a propeller mixer. Then add phase B to phase A. Stir slowly until the mixture is homogeneous. Thoroughly homogenize phase C until the pigments are well distributed. Add phase C and phase D to phase AB and mix thoroughly.

### Application Example 13

### Waterproof Mascara

| A | |
|---|---|
| 46.7 | dist. water |
| 3.0 | Lutrol E 400 (PEG-8) |
| 0.5 | xanthan gum |
| q.s. | preservative |
| 0.1 | imidazolidinylurea |
| 1.3 | tetrahydroxypropylethylenediamine |
| B | |
| 8.0 | carnauba wax |
| 4.0 | beeswax |
| 4.0 | iscoeicosane |
| 4.0 | polyisobutene |
| 5.0 | stearic acid |
| 1.0 | glyceryl stearate |
| q.s. | preservative |
| 2.0 | benzyl laurate |
| C | |
| 10.0 | iron oxide/Al powder/silica (e.g. Sicopearl Fantastico |
| | Gold ™ from BASF) |
| E | |
| 8.0 | polyurethane-1 |
| 2.0 | polymer according to preparation example 38 |

Preparation:

Heat phase A and phase B separately to 85° C. Maintain the temperature and add phase C to phase A and homogenize until the pigments are uniformly distributed. Add phase B to phase AC and homogenize for 2-3 minutes. Then add phase E and stir slowly. Cool to room temperature.

### Application Example 14

### Sun Protection Gel

| Phase A | |
|---|---|
| 1.00 | PEG-40 hydrogenated castor oil |
| 8.00 | octyl methoxycinnamate (Uvinul MC 80 ™ from BASF) |
| 5.00 | octocrylene (Uvinul N 539 ™ from BASF) |
| 0.80 | octyl triazone (Uvinul T 150 ™ from BASF) |
| 2.00 | butylmethoxydibenzoylmethane (Uvinul BMBM ™ from BASF) |
| 2.00 | tocopheryl acetate |
| q.s. | perfume oil |
| Phase B | |
| 2.50 | polymer according to preparation example 6 |
| 0.30 | acrylates/C10-30 alkyl acrylate crosspolymer |
| 0.20 | carbomer |
| 5.00 | glycerol |
| 0.20 | disodium EDTA |
| q.s. | preservative |
| 72.80 | dist. water |
| Phase C | |
| 0.20 | sodium hydroxide |

Preparation:

Mix the components of phase A. Allow phase B to swell and stir into phase A with homogenization. Neutralize with phase C and homogenize again.

### Preparation Example 15

### Sun Protection Emulsion Containing TiO₂ and ZnO₂

| | |
|---|---|
| Phase A | |
| 6.00 | PEG-7 hydrogenated castor oil |
| 2.00 | PEG-45/dodecyl glycol copolymer |
| 3.00 | isopropyl myristate |
| 8.00 | jojoba (Buxus chinensis) oil |
| 4.00 | octyl methoxycinnamate (Uvinul MC 80) |
| 2.00 | 4-methylbenzylidenecamphor (Uvinul MBC 95) |
| 3.00 | titanium dioxide, dimethicone |
| 1.00 | dimethicone |
| 5.00 | zinc oxide, dimethicone |
| Phase B | |
| 2.00 | polymer according to preparation example 20 |
| 0.20 | disodium EDTA |
| 5.00 | glycerol |
| q.s. | preservative |
| 58.80 | dist. water |
| Phase C | |
| q.s. | perfume oil |

Preparation:

Heat phases A and B separately to about 85° C. Stir phase B into phase A and homogenize. Cool to about 40° C., add phase C and briefly homogenize again.

### Application Example 16

### Sun Protection Lotion

| | |
|---|---|
| Phase A | |
| 6.00 | octyl methoxycinnamate (Uvinul MC 80 ™ from BASF) |
| 2.50 | 4-methylbenzylidenecamphor (Uvinul MBC 95 ™ from BASF) |
| 1.00 | octyltriazone (Uvinul T 150 ™ from BASF) |
| 2.00 | butylmethoxydibenzoylmethane (Uvinul BMBM ™ from BASF) |
| 2.00 | PVP/hexadecene copolymer |
| 5.00 | PPG-3 myristyl ether |
| 0.50 | dimethicone |
| 0.10 | BHT, ascorbyl palmitate, citric acid, glyceryl stearate, |
| | propylene glycol |
| 2.00 | cetyl alcohol |
| 2.00 | potassium cetyl phosphate |
| Phase B | |
| 2.50 | polymer according to preparation example 25 |
| 5.00 | propylene glycol |
| 0.20 | disodium EDTA |
| q.s. | preservative |
| 63.92 | dist. water |
| Phase C | |
| 5.00 | mineral oil |
| 0.20 | carbomer |
| Phase D | |
| 0.08 | sodium hydroxide |
| Phase E | |
| q.s. | perfume oil |

Preparation:

Heat phases A and B separately to about 80° C. Stir phase B into phase A with homogenization; briefly afterhomogenize.

Slurry phase C, stir into phase AB, neutralize with phase D and afterhomogenize. Cool to about 40° C., introduce phase E, homogenize again.

### Application Example 17

### Removable Face Mask

| Phase A | |
|---|---|
| 57.10 | dist. water |
| 6.00 | polyvinyl alcohol |
| 5.00 | propylene glycol |
| Phase B | |
| 20.00 | alcohol |
| 4.00 | PEG-32 |
| q.s | perfume oil |
| Phase C | |
| 5.00 | polyquaternium-44 |
| 2.70 | polymer according to preparation example 19 |
| 0.20 | allantoin |

Preparation:

Heat phase A to at least 90° C. and stir until dissolved. Dissolve phase B at 50° C. and stir into phase A. At about 35° C., compensate the ethanol loss. Add phase C and stir.

Application Example 18: Face mask
Phase A

| | |
|---|---|
| 3.00 | ceteareth-6 |
| 1.50 | ceteareth-25 |
| 5.00 | cetearyl alcohol |
| 6.00 | cetearyl octanoate |
| 6.00 | mineral oil |
| 0.20 | bisabolol |
| 3.00 | glyceryl stearate |

Phase B

| | |
|---|---|
| 2.00 | propylene glycol |
| 5.00 | panthenol |
| 2.80 | polymer according to preparation example 2 |
| q.s. | preservative |
| 65.00 | dist. water |

Phase C

| | |
|---|---|
| q.s. | perfume oil |
| 0.50 | tocopheryl acetate |

Preparation:

Heat phase A and B separately to about 80° C. Stir phase B into phase A with homogenization, briefly afterhomogenize. Cool to about. 40° C., add phase C, homogenize again.

### Application Example 19

### Body Lotion Foam

Phase A

| | |
|---|---|
| 1.50 | Ceteareth-25 |
| 1.50 | Ceteareth-6 |
| 4.00 | cetearyl alcohol |
| 10.00 | cetearyl octanoate |
| 1.00 | dimethicone |

Phase B

| | |
|---|---|
| 3.00 | polymer according to preparation example 9 |
| 2.00 | panthenol |
| 2.50 | propylene glycol |
| q.s. | preservative |
| 74.50 | dist. water |

Phase C

| | |
|---|---|
| q.s. | perfume oil |

Preparation:

Heat phases A and B separately to about 80° C. Stir phase B into phase A and homogenize. Cool to about 40° C., add phase C and briefly homogenize again. Containerizing: 90% of active ingredient and 10% of propane/butane at 3.5 bar (20° C.).

### Application Example 20

### Face Wash for Dry and Sensitive Skin

Phase A

| | |
|---|---|
| 2.50 | PEG-40 hydrogenated castor oil |
| q.s. | perfume oil |
| 0.40 | bisabolol |

Phase B

| | |
|---|---|
| 3.00 | glycerol |
| 1.00 | hydroxyethyl cetyldimonium phosphate |
| 5.00 | witch hazel (Hamamelis virginiana) distillate |
| 0.50 | panthenol |
| 0.50 | polymer according to preparation example 3 |
| q.s. | preservative |
| 87.60 | dist. water |

Preparation:

Dissolve phase A until clear. Stir phase B into phase A.

### Application Example 21

### Face Wash Paste with Peeling Effect

Phase A

| | |
|---|---|
| 70.00 | dist. water |
| 3.00 | polymer according to preparation example 3 |
| 1.50 | carbomer |
| q.s. | preservative |

Phase B

| | |
|---|---|
| q.s. | perfume oil |
| 7.00 | potassium cocoyl hydrolyzed protein |
| 4.00 | cocamidopropylbetaine |

Phase C

| | |
|---|---|
| 1.50 | triethanolamine |

Phase D

| | |
|---|---|
| 13.00 | polyethylene (Luwax A ™from BASF) |

Preparation:

Allow phase A to swell. Dissolve phase B until clear. Stir phase B into phase A. Neutralize with phase C. Then stir in phase D.

### Application Example 22

### Face Soap

Phase A

| | |
|---|---|
| 25.0 | potassium cocoate |
| 20.0 | disodium cocoamphodiacetate |
| 2.0 | lauramide DEA |
| 1.0 | glycol stearate |
| 2.0 | polymer according to preparation example 33 |
| 50.0 | dist. water |
| q.s. | citric acid |

Phase B

| | |
|---|---|
| q.s. | preservative |
| q.s. | perfume oil |

Preparation:

Heat phase A to 70° C. with stirring until homogeneous. Adjust pH to 7.0 to 7.5 with citric acid. Cool to 50° C. and add phase B.

### Application Example 23

### Face Cleansing Milk O/W Type

Phase A

| | |
|---|---|
| 1.50 | ceteareth-6 |
| 1.50 | ceteareth-25 |
| 2.00 | glyceryl stearate |
| 2.00 | cetyl alcohol |
| 10.00 | mineral oil |

Phase B

| | |
|---|---|
| 5.00 | propylene glycol |
| q.s. | preservative |
| 1.0 | polymer according to preparation example 12 |
| 66.30 | dist. water |

Phase C

| | |
|---|---|
| 0.20 | carbomer |
| 10.00 | cetearyl octanoate |

Phase D

| | |
|---|---|
| 0.40 | tetrahydroxypropylethylenediamine |

Phase E

| | |
|---|---|
| q.s. | perfume oil |
| 0.10 | bisabolol |

Preparation:

Heat phases A and B separately to about 80° C. Stir phase B into phase A with homogenization, and briefly afterhomogenize. Slurry phase C, stir into phase AB, neutralize with phase D and afterhomogenize. Cool to about 40° C., add phase E, homogenize again.

### Application Example 24

### Transparent Soap

| | |
|---|---|
| 4.20 | sodium hydroxide |
| 3.60 | dist. water |
| 2.0 | polymer according to preparation example 14 |
| 22.60 | propylene glycol |
| 18.70 | glycerol |
| 5.20 | cocoamide DEA |
| 10.40 | cocamine oxide |
| 4.20 | sodium lauryl sulfate |
| 7.30 | myristic acid |
| 16.60 | stearic acid |
| 5.20 | tocopherol |

Preparation:

Mix all ingredients. Melt the mixture at 85° C. until clear. Immediately pour into the mold.

### Application Example 25

### Peeling Cream, O/W Type

Phase A

| | |
|---|---|
| 3.00 | ceteareth-6 |
| 1.50 | ceteareth-25 |
| 3.00 | glyceryl stearate |
| 5.00 | cetearyl alcohol, sodium cetearyl sulfate |
| 6.00 | cetearyl octanoate |
| 6.00 | mineral oil |
| 0.20 | bisabolol |

Phase B

| | |
|---|---|
| 2.00 | propylene glycol |
| 0.10 | disodium EDTA |
| 3.00 | polymer according to preparation example 18 |
| q.s. | preservative |
| 59.70 | dist. water |

Phase C

| | |
|---|---|
| 0.50 | tocopheryl acetate |
| q.s. | perfume oil |

Phase D

| | |
|---|---|
| 10.00 | polyethylene |

Preparation:

Heat phases A and B separately to about 80° C. Stir phase B into phase A and homogenize. Cool to about 40° C., add phase C and briefly homogenize again. Then stir in phase D.

### Application Example 26

### Shaving Foam

| | |
|---|---|
| 6.00 | ceteareth-25 |
| 5.00 | poloxamer 407 |
| 52.00 | dist. water |
| 1.00 | triethanolamine |
| 5.00 | propylene glycol |
| 1.00 | PEG-75 lanolin oil |
| 5.00 | polymer according to preparation example 5 |
| q.s. | preservative |
| q.s. | perfume oil |
| 25.00 | sodium laureth sulfate |

Preparation:

Weigh everything together, then stir until dissolved. Containerizing: 90 parts of active substance and 10 parts of 25:75 propane/butane mixture.

### Application Example 27

### Aftershave Balsam

Phase A

| | |
|---|---|
| 0.25 | acrylates/C10-30 alkyl acrylate crosspolymer |
| 1.50 | tocopheryl acetate |
| 0.20 | bisabolol |
| 10.00 | caprylic/capric triglyceride |
| q.s. | perfume oil |
| 1.00 | PEG-40 hydrogenated castor oil |

Phase B

| | |
|---|---|
| 1.00 | panthenol |
| 15.00 | alcohol |
| 5.00 | glycerol |
| 0.05 | hydroxyethylcellulose |
| 1.92 | polymer according to preparation example 8 |
| 64.00 | dist. water |

Phase C

| | |
|---|---|
| 0.08 | sodium hydroxide |

Preparation:

Mix the components of phase A. Stir phase B into phase A with homogenization, then briefly afterhomogenize. Neutralize with phase C and homogenize again.

### Application Example 28

### Bodycare Cream

| | |
|---|---|
| Phase A | |
| 2.00 | ceteareth-6 |
| 2.00 | ceteareth-25 |
| 2.00 | cetearyl alcohol |
| 3.00 | glyceryl stearate SE |
| 5.00 | mineral oil |
| 4.00 | jojoba (Buxus chinensis) oil |
| 3.00 | cetearyl octanoate |
| 1.00 | dimethicone |
| 3.00 | mineral oil, lanolin alcohol |
| Phase B | |
| 5.00 | propylene glycol |
| 0.50 | veegum |
| 1.00 | panthenol |
| 1.70 | polymer according to preparation example 14 |
| 6.00 | polyquaternium-44 |
| q.s. | preservative |
| 60.80 | dist. water |
| Phase C | |
| q.s. | perfume oil |

Preparation:

Heat phases A and B separately to about 80° C. Homogenize phase B. Stir phase B into phase A with homogenization, then briefly afterhomogenize.

Cool to about 40° C., add phase C and briefly homogenize again.

### Application Example 29

### Toothpaste

Phase A

| | |
|---|---|
| 34.79 | dist. water |
| 3.00 | polymer according to preparation example 13 |
| 0.30 | preservative |
| 20.00 | glycerol |
| 0.76 | sodium monofluorophosphate |

Phase B

| | |
|---|---|
| 1.20 | sodium carboxymethylcellulose |

Phase C

| | |
|---|---|
| 0.80 | aroma oil |
| 0.06 | saccharin |
| 0.10 | preservative |
| 0.05 | bisabolol |
| 1.00 | panthenol |
| 0.50 | tocopheryl acetate |
| 2.80 | silica |
| 1.00 | sodium lauryl sulfate |
| 7.90 | dicalcium phosphate anhydrate |
| 25.29 | dicalcium phosphate dihydrate |
| 0.45 | titanium dioxide |

Preparation:

Dissolve phase A. Spread phase B into phase A and dissolve. Add phase C and stir under reduced pressure at RT for about 45 min.

### Application Example 30

### Mouthwash

Phase A

| | |
|---|---|
| 2.00 | aroma oil |
| 4.00 | PEG-40 hydrogenated castor oil |
| 1.00 | bisabolol |
| 30.00 | alcohol |

Phase B

| | |
|---|---|
| 0.20 | saccharin |
| 5.00 | glycerol |
| q.s. | preservative |
| 5.00 | poloxamer 407 |
| 0.5 | polymer according to preparation example 7 |
| 52.30 | dist. water |

Preparation:

Dissolve phase A and phase B separately until clear. Stir phase B into phase A.

### Application Example 31

### Denture Adhesive

Phase A

| | |
|---|---|
| 0.20 | bisabolol |
| 1.00 | beta-carotene |
| q.s. | aroma oil |
| 20.00 | cetearyl octanoate |
| 5.00 | silica |
| 33.80 | mineral oil |

Phase B

| | |
|---|---|
| 5.00 | polymer according to preparation example 37 |
| 35.00 | PVP (20% strength solution in water) |

Preparation:

Slowly mix phase A. Stir phase B into phase A.

### Application Example 32

### Skincare Cream, O/W Type

Phase A

| | |
|---|---|
| 8.00 | cetearyl alcohol |
| 2.00 | ceteareth-6 |
| 2.00 | ceteareth-25 |
| 10.00 | mineral oil |
| 5.00 | cetearyl octanoate |
| 5.00 | dimethicone |

Phase B

| | |
|---|---|
| 3.00 | polymer according to preparation example 19 |
| 2.00 | panthenol, propylene glycol |
| q.s. | preservative |
| 63.00 | dist. water |

Phase C

| | |
|---|---|
| q.s. | perfume oil |

Preparation:

Heat phase A and B separately to about 80° C. Stir phase B into phase A with homogenization, then briefly afterhomogenize. Cool to about 40° C., add phase C, homogenize again.

### Application Example 33

### Skincare Cream, W/O Type

Phase A

| | |
|---|---|
| 6.00 | PEG-7 hydrogenated castor oil |
| 8.00 | cetearyl octanoate |
| 5.00 | isopropyl myristate |
| 15.00 | mineral oil |
| 2.00 | PEG-45/dodecyl glycol copolymer |
| 0.50 | magnesium stearate |
| 0.50 | aluminum stearate |

Phase B

| | |
|---|---|
| 3.00 | glycerol |
| 3.30 | polymer according to preparation example 12 |
| 0.70 | magnesium sulfate |
| 2.00 | panthenol |
| q.s. | preservative |
| 48.00 | dist. water |

Phase C

| | |
|---|---|
| 1.00 | tocopherol |
| 5.00 | tocopheryl acetate |
| q.s. | perfume oil |

Preparation:

Heat phases A and B separately to about 80° C. Stir phase B into phase A and homogenize. Cool to about 40° C., add phase C and briefly homogenize again.

### Application Example 34

### Lipcare Cream

Phase A

| | |
|---|---|
| 10.00 | cetearyl octanoate |
| 5.00 | polybutene |

Phase B

| | |
|---|---|
| 0.10 | carbomer |

Phase C

| | |
|---|---|
| 2.00 | ceteareth-6 |
| 2.00 | ceteareth-25 |
| 2.00 | glyceryl stearate |
| 2.00 | cetyl alcohol |
| 1.00 | dimethicone |
| 1.00 | benzophenone-3 |
| 0.20 | bisabolol |
| 6.00 | mineral oil |

Phase D

| | |
|---|---|
| 8.00 | polymer according to preparation example 16 |
| 3.00 | panthenol |
| 3.00 | propylene glycol |
| q.s. | preservative |
| 54.00 | dist. water |

Phase E

| | |
|---|---|
| 0.10 | triethanolamine |

Phase F

| | |
|---|---|
| 0.50 | tocopheryl acetate |
| 0.10 | tocopherol |
| q.s. | perfume oil |

Preparation:

Dissolve phase A until clear. Add phase B and homogenize. Add phase C and melt at 80° C. Heat phase D to 80° C. Add phase D to phase ABC and homogenize. Cool to about 40° C., add phase E and phase F, homogenize again.

### Application Example 35

### Glossy Lipstick

Phase A

| | |
|---|---|
| 5.30 | candelilla (Euphorbia cerifera) wax |
| 1.10 | beeswax |
| 1.10 | microcrystalline wax |
| 2.00 | cetyl palmitate |
| 3.30 | mineral oil |
| 2.40 | castor oil, glyceryl ricinoleate, octyldodecanol, carnauba, |
| | candelilla wax, |
| 0.40 | bisabolol |
| 16.00 | cetearyl octanoate |
| 2.00 | hydrogenated cocoglycerides |
| q.s. | preservative |
| 1.00 | polymer according to preparation example 39 |
| 60.10 | castor (Ricinus communis) oil |
| 0.50 | tocopheryl acetate |

Phase B

| | |
|---|---|
| 0.80 | C. I. 14 720:1, Acid Red 14 aluminum lake |

Phase C

| | |
|---|---|
| 4.00 | mica, titanium dioxide |

Preparation:

Weigh in the components of phase A and melt. Incorporate phase B until homogeneous. Add phase C and stir in. Cool to room temperature with stirring.

We claim:

1. The use of polymers obtainable by free-radical polymerization of
a) at least one vinyl ester of C₁-C₂₄-carboxylic acids in the presence of
b) polyether-containing compounds having a molecular weight of at least 300, and
c) one or more further copolymerizable monomers chosen from the group of monomers consisting of methyl methacrylate, N-vinylpyrrolidone, 3-methyl-1-vinylimidazolium methylsulfate, N-vinylformamide, pentaerythritol triallyl ether and N,N′-divinylethyleneurea,
in skin cosmetic formulations and in decorative cosmetics.

2. The use as claimed in claim 1, wherein, after the polymerization, the ester functions of the original monomers a) are at least partially hydrolyzed.

3. The use as claimed in claim 1 and 2, wherein the polymers are obtainable by free-radical polymerization of
a) at least one vinyl ester of C₁-C₂₄-carboxylic acids in the presence of
b) polyether-containing compounds of the formula I 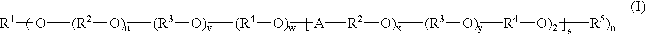
in which the variables independently of one another have the following meanings:
R¹ is hydrogen, C₁-C₂₄-alkyl, R⁶—C(═O)—, R⁶—NH—C(═O)—, polyalcohol radical;
R⁵ is hydrogen, C₁-C₂₄-alkyl, R⁶—C(═O)—, R⁶—NH—C(═O)—;
R² to R⁴ are
—(CH₂)₂—, —(CH₂)₃—, —(CH₂)₄—, —CH₂—CH(R⁶)—, —CH₂—CHOR⁷—CH₂—;
R⁶ is C₁-C₂₄-alkyl;
R⁷ is hydrogen, C₁-C₂₄-alkyl, R⁶—C(═O)—, R⁶—NH—C(═O)—;
A is —C(═O)—O, —C(═O)—B—C(═O)—O, —C(═O)—NH—B—NH—C(═O)—O;
B is —(CH₂)ₜ—, arylene, optionally substituted;
n is 1 to 1 000;
s is 0 to 1 000;
t is 1 to 12;
u is 1 to 5 000;
v is 0 to 5 000;
w is 0 to 5 000;
x is 0 to 5 000;
y is 0 to 5 000;
z is 0 to 5 000; and
c) one or more further copolymerizable monomers chosen from the group of monomers consisting of methyl methacrylate, N-vinylpyrrolidone, 3-methyl-1-vinylimidazolium methylsulfate, N-vinylformamide, pentaerythritol triallyl ether and N,N′-divinylethyleneurea.

4. The use of polymers as claimed in claim 1, wherein the polymers are obtainable by free-radical polymerization of
a) at least one vinyl ester of C₁-C₂₄-carboxylic acids in the presence of
b) polyether-containing compounds of the formula I having an average molecular weight of from 300 to 100 000 (according to the number average), in which the variables independently of one another have the following meanings:
R¹ is hydrogen, C₁-C₁₂-alkyl, R⁶—C(═O)—, R⁶—NH—C(═O)—, polyalcohol radical;
R⁵ is hydrogen, C₁-C₁₂-alkyl, R⁶—C(═O)—, R⁶—NH—C(═O)—;
R² to R⁴ are
—(CH₂)₂—, —(CH₂)₃—, —(CH₂)₄—, —CH₂—CH(R⁶)—, —CH₂—CHOR⁷—CH₂—;
R⁶ is C₁-C₁₂-alkyl;
R⁷ is hydrogen, C₁-C₁₂-alkyl, R⁶—C(═O)—, R⁶—NH—C(═O)—;
n is 1 to 8;
s is 0;
u is 2 to 2 000;
v is 0 to 2 000;
w is 0 to 500; and
c) one or more further copolymerizable monomers chosen from the group of monomers consisting of methyl methacrylate, N-vinylpyrrolidone, 3-methyl-1-vinylimidazolium methylsulfate, N-vinylformamide, pentaerythritol triallyl ether and N,N′-divinylethyleneurea.

5. The use of polymers as claimed in claim 1, wherein the polymers are obtainable by free-radical polymerization of
a) at least one vinyl ester of C₁-C₂₄-carboxylic acids in the presence of
b) polyether-containing compounds of the formula I having an average molecular weight of from 500 to 50 000 (according to the number average), in which the variables independently of one another have the following meaning:
R¹ is hydrogen, C₁-C₆-alkyl, R⁶—C(═O)—, R⁶—NH—C(═O)—;
R⁵ is hydrogen, C₁-C₆-alkyl, R⁶—C(═O)—, R⁶—NH—C(═O)—;
R² to R⁴ are
—(CH₂)₂—, —(CH₂)₃—, —(CH₂)₄—, —CH₂—CH(R⁶)—, —CH₂—CHOR⁷—CH₂—;
R⁶ is C₁-C₆-alkyl;
R⁷ is hydrogen, C₁-C₆-alkyl, R⁶—C(═O)—, R⁶—NH—C(═O)—;
n is 1;
s is 0;
u is 5 to 500;
v is 0 to 500;
w is 0 to 500; and
c) one or more further copolymerizable monomers chosen from the group of monomers consisting of methyl methacrylate, N-vinylpyrrolidone, 3-methyl-1-vinylimidazolium methylsulfate, N-vinylformamide, pentaerythritol triallyl ether and N,N′-divinylethyleneurea, in skin cosmetic formulations.

6. The use of polymers as claimed in claim 1, wherein the polymers are obtainable by free-radical polymerization of
a) at least one vinyl ester of C₁-C₂₄-carboxylic acids in the presence of
b) polyether-containing silicone derivatives and
c) one or more further copolymerizable monomers chosen from the group of monomers consisting of methyl methacrylate, N-vinylpyrrolidone, 3-methyl-1-vinylimidazolium methylsulfate, N-vinylformamide, pentaerythritol triallyl ether and N,N′-divinylethyleneurea.

7. The use of polymers as claimed in claim 6, wherein the polymers are obtainable by free-radical polymerization of
a) at least one vinyl ester of C₁-C₂₄-carboxylic acids in the presence of
b) polyether-containing silicone derivatives of the formula II 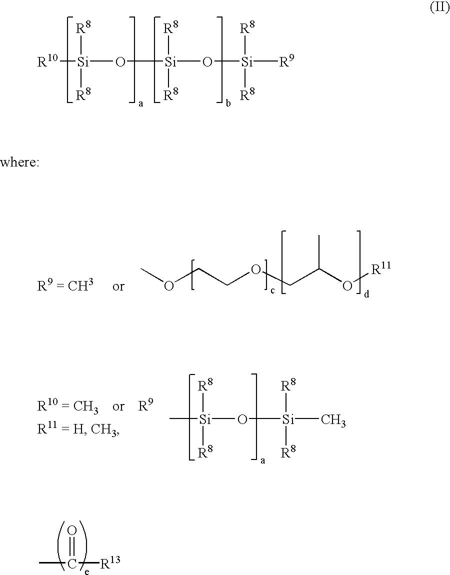
R¹³ is a C₁-C₄₀ organic radical which can contain amino, carboxylic acid or sulfonate groups, or for the case e=0, is also the anion of an inorganic acid,
and where the radicals R⁸ can be identical or different, and either originate from the group of aliphatic hydrocarbons having 1 to 20 carbon atoms, are cyclic aliphatic hydrocarbons having 3 to 20 carbon atoms, are of an aromatic nature or are identical to R¹², where: 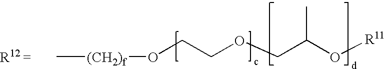
with the proviso that at least one of the radicals R⁸, R⁹ or R¹⁰ is a polyalkylene oxide-containing radical as defined above,
and f is an integer from 1 to 6,
a and b are integers such that the molecular weight of the polysiloxane block is between 300 and 30 000,
c and d can be integers between 0 and 50, with the proviso that the sum c+d is greater than 0, and e is 0 or 1,
and
one or more further copolymerizable monomers chosen from the group of monomers consisting of methyl methacrylate, N-vinylpyrrolidone, 3-methyl-1-vinylimidazolium methylsulfate, N-vinylformamide, pentaerythritol triallyl ether and N,N′-divinylethyleneurea.

8. The use of polymers as claimed in claim 7, wherein formula II has the following meaning:

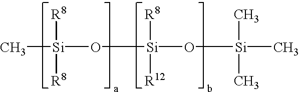

9. The use as claimed in claim 1, wherein the polymers are obtainable by free-radical polymerization of
a) at least one vinyl ester of C₁-C₂₄-carboxylic acids in the presence of
b) polyether-containing compounds obtainable by reaction of polyethyleneimines with alkylene oxides
and
c) one or more further copolymerizable monomers chosen from the group of monomers consisting of methyl methacrylate, N-vinylpyrrolidone, 3-methyl-1-vinylimidazolium methylsulfate, N-vinylformamide, pentaerythritol triallyl ether and N,N′-divinylethyleneurea.

10. The use of polymers as claimed in claim 9, wherein the alkylene oxides are ethylene oxide, propylene oxide, butylene oxide and mixtures thereof.

11. The use of polymers as claimed in claims 9 and 10, wherein the alkylene oxide is ethylene oxide.

12. The use of polymers as claimed in claims 9, 10 and 11, wherein the polyethyleneimine has a molecular weight between 300 and 20 000.

13. The use of polymers as claimed in claim 1, wherein the polyether-containing compounds b) have been prepared by polymerization of ethylenically unsaturated alkylene oxide-containing monomers and optionally further copolymerizable monomers.

14. The use of polymers as claimed in claim 13, wherein the polyether-containing compounds b) have been prepared by polymerization of polyalkylene oxide vinyl ethers and optionally further copolymerizable monomers.

15. The use of polymers as claimed in claim 13, wherein the polyether-containing compounds b) have been prepared by polymerization of polyalkylene oxide (meth)acrylates and optionally further copolymerizable monomers.

16. The use of polymers as claimed in claim 1, wherein the quantitative ratios are

| | |
|---|---|
| a) | 10-98% by weight |
| b) | 2-90% by weight |
| c) | up to 50% by weight. |

17. The use of polymers as claimed in claims 1 to 15, wherein the quantitative ratios are

| | |
|---|---|
| a) | 50-97% by weight |
| b) | 3-50% by weight |
| c) | up to 30% by weight. |

18. The use of polymers as claimed in claims 1 to 15, wherein the quantitative ratios are

| | | |
|---|---|---|
| a) | 65-97% | by weight |
| b) | 3-35% | by weight |
| c) | up to 20% | by weight. |

19. A skin cosmetic formulation which has the following composition:

| | | |
|---|---|---|
| a) | 0.05-20% | by weight of the polymer as in claim 1 |
| b) | 20-99.95% | by weight of water and/or solvents and/or an |
| | | oil component |
| c) | 0-79.5% | by weight of further constituents. |

20. A skin cosmetic formulation as claimed in claim 19 which is an emulsion and has the following composition:

| | | |
|---|---|---|
| a) | 0.05-10% | by weight of the polymer as in claim 1 |
| b) | 10-94.94% | by weight of water |
| c) | 5-89.94% | by weight of an oil component |
| d) | 0.01-40% | by weight of an emulsifier |
| e) | 0-74.94% | by weight of further constituents. |

21. A skin cosmetic formulation as claimed in claims 19 and 20 which is a W/O emulsion and has the following composition:

| | | |
|---|---|---|
| a) | 0.05-10% | by weight of the polymer as in claim 1 |
| b) | 20-77.95% | by weight of water |
| c) | 20-77.95% | by weight of an oil component |
| d) | 2-35% | by weight of an emulsifier |
| e) | 0-55.95% | by weight of further constituents. |

22. A skin cosmetic formulation as claimed in claims 19 and 20 which is an O/W emulsion and has the following composition:

| | | |
|---|---|---|
| a) | 0.05-10% | by weight of the polymer as in claim 1 |
| b) | 40-96.95% | by weight of water |
| c) | 1-44.95% | by weight of an oil component |
| d) | 1-35% | by weight of an emulsifier |
| e) | 0-10% | by weight of a gel former |
| f) | 0-57.95% | by weight of further constituents. |
